SEQUENCE LISTING (38 sequences)

SEQ ID NO: 1 - protein, 502 aa, Streptococcus agalactiae
MKKKMIQSLLVASLAFGMAVSPVTPIAFAAETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTELPSTGGIGTTIFYIIGAILVIGAGIVLVARRRLRS

SEQ ID NO: 2 - protein, 438 aa, Streptococcus agalactiae
AETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTE

SEQ ID NO: 3 - protein, 554 aa, Streptococcus agalactiae
MKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTGGIGTAIFVAIGAAVMAFAVKGMKRRTKDN

SEQ ID NO: 4 - protein, 483 aa, Streptococcus agalactiae
AEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 5 - protein, 271 aa, Streptococcus agalactiae
AEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKG

SEQ ID NO: 6 - protein, 14 aa, Artificial Sequence
  Linker
GGGGSGGGGSGGGG

SEQ ID NO: 7 - protein, 17 aa, Artificial Sequence
  Linker
GGGGSGGGGSGGGGSEL

SEQ ID NO: 8 - protein, 3 aa, Artificial Sequence
  N-terminal sequence
MAS

SEQ ID NO: 9 - protein, 6 aa, Artificial Sequence
  C-terminal sequence
HHHHHH

SEQ ID NO: 10 - protein, 941 aa, Artificial Sequence
  RH1
MASAETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTEGGGGSGGGGSGGGGSELAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 11 - DNA, 2826 bp, Artificial Sequence
  RH1
atggctagcgctgagacagggacaattacagttcaagatactcaaaaaggcgcaacctataaagcatataaagtttttgatgcagaaatagataatgcaaatgtatctgattcgaataaagatggagcttcttatttaattcctcaaggtaaagaagctgagtataaagcttcaactgattttaattctctttttacgacaactactaatggagggagaacatatgtaactaaaaaagatactgcgtcagcaaatgagattgcgacatgggctaaatctatatcagctaatactacaccagtttccactgttactgagtcaaataatgatggtactgaggttattaatgtttcccaatatggatattattatgtttctagcactgttaataatggagctgtgattatggttacatctgtaactccaaatgctactattcatgaaaagaatactgatgcgacatggggagatggtggtggaaaaactgtagatcaaaaaacgtactcggttggtgatacagtcaaatatactattacttataagaatgcagtcaattatcatggtacagaaaaagtgtatcaatatgttataaaggatactatgccatctgcttctgtagttgatttgaacgaagggtcttatgaagtaactattactgatggatcagggaatattacaactctaactcaaggttcggaaaaagcaactgggaagtataacctgttagaggaaaataataatttcacgattactattccgtgggcagctaccaatactccaaccggaaatactcaaaatggagctaatgatgactttttttataagggaataaatacaatcacagtcacttatacaggagtattaaagagtggagctaaaccaggttcagctgatttaccagaaaatacaaacattgcgaccatcaaccccaatactagcaatgatgacccaggtcaaaaagtaacagtgagggatggtcaaattactataaaaaaaattgatggttccacaaaagcttcattacaaggtgctatatttgttttaaagaatgctacgggtcaatttctaaactttaacgatacaaataacgttgaatggggcacagaagctaatgcaacagaatatacaacaggagcagatggtataattaccattacaggcttgaaagaaggtacatactatctagttgagaaaaaggctcccttaggttacaatttgttagataactctcagaaggttattttaggagatggagccactgatacgactaattcagataaccttttagttaacccaactgttgaaaataacaaaggtactgagggtggcggaggctccggcggaggcggttctggtggcggaggcagcgagctcgcagaagtgtcacaagaacgcccagcgaaaacaacagtaaatatctataaattacaagctgatagttataaatcggaaattacttctaatggtggtatcgagaataaagacggcgaagtaatatctaactatgctaaacttggtgacaatgtaaaaggtttgcaaggtgtacagtttaaacgttataaagtcaagacggatatttctgttgatgaattgaaaaaattgacaacagttgaagcagcagatgcaaaagttggaacgattcttgaagaaggtgtcagtctacctcaaaaaactaatgctcaaggtttggtcgtcgatgctctggattcaaaaagtaatgtgagatacttgtatgtagaagatttaaagaattcaccttcaaacattaccaaagcttatgctgtaccgtttgtgttggaattaccagttgctaactctacaggtacaggtttcctttctgaaattaatatttaccctaaaaacgttgtaactgatgaaccaaaaacagataaagatgttaaaaaattaggtcaggacgatgcaggttatacgattggtgaagaattcaaatggttcttgaaatctacaatccctgccaatttaggtgactatgaaaaatttgaaattactgataaatttgcagatggcttgacttataaatctgttggaaaaatcaagattggttcgaaaacactgaatagagatgagcactacactattgatgaaccaacagttgataaccaaaatacattaaaaattacgtttaaaccagagaaatttaaagaaattgctgagctacttaaaggaatgacccttgttaaaaatcaagatgctcttgataaagctactgcaaatacagatgatgcggcatttttggaaattccagttgcatcaactattaatgaaaaagcagttttaggaaaagcaattgaaaatacttttgaacttcaatatgaccatactcctgataaagctgacaatccaaaaccatctaatcctccaagaaaaccagaagttcatactggtgggaaacgatttgtaaagaaagactcaacagaaacacaaacactaggtggtgctgagtttgatttgttggcttctgatgggacagcagtaaaatggacagatgctcttattaaagcgaatactaataaaaactatattgctggagaagctgttactgggcaaccaatcaaattgaaatcacatacagacggtacgtttgagattaaaggtttggcttatgcagttgatgcgaatgcagagggtacagcagtaacttacaaattaaaagaaacaaaagcaccagaaggttatgtaatccctgataaagaaatcgagtttacagtatcacaaacatcttataatacaaaaccaactgacatcacggttgatagtgctgatgcaacacctgatacaattaaaaacaacaaacgtccttcataa

SEQ ID NO: 12 - DNA, 12 bp, Artificial Sequence
  Shine-Dalgarno sequence in wild-type SpbI
taatggagctgt

SEQ ID NO: 13 - DNA, 31 bp, Artificial Sequence
  Primer
gggaattcgctagcgcagaagtgtcacaaga

SEQ ID NO: 14 - DNA, 67 bp, Artificial Sequence
  Primer
gagctcgctgcctccgccaccagaaccgcctccgccggagcctccgccacctgaaggacgtttgttg

SEQ ID NO: 15 - DNA, 70 bp, Artificial Sequence
  Primer
gagctcgctgcctccgccaccagaaccgcctccgccggagcctccgccaccctgagacagggacaattac

SEQ ID NO: 16 - DNA, 34 bp, Artificial Sequence
  Primer
gcgtctcgagctcagtacctttgttattttcaac

SEQ ID NO: 17 - DNA, 15 bp, Artificial Sequence
  RBS2 in wild-type SpbI
ggagctgtgattatg

SEQ ID NO: 18 - DNA, 15 bp, Artificial Sequence
  Mutated RBS2 in SpbI
ggtgctgtgattatg

SEQ ID NO: 19 - protein, 26 aa, Artificial Sequence
  Adjuvant
ICICICICICICICICICICICICIC

SEQ ID NO: 20 - protein, 11 aa, Artificial Sequence
  Adjuvant
KLKLLLLLKLK

SEQ ID NO: 21 - protein, 4 aa, Artificial Sequence
  Linker
GGGG

SEQ ID NO: 22 - protein, 901 aa, Streptococcus agalactiae
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGIIPMTGGKGILSFILIGGAMMSIAGGIYIWKRYKKSSDMSIKKD

SEQ ID NO: 23 - protein, 869 aa, Streptococcus agalactiae
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGIIPMTGGKGILS

SEQ ID NO: 24 - protein, 858 aa, Streptococcus agalactiae
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGI

SEQ ID NO: 25 - protein, 6 aa, Artificial Sequence
  Linker
GSGGGG

SEQ ID NO: 26 - protein, 932 aa, Artificial Sequence
  RH2
MASAETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTEGSGGGGELAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 27 - DNA, 57 bp, Artificial Sequence
  Primer
agcgagtttagctagcgctgagacagggacaattacagttcaagatactcaaaaagg

SEQ ID NO: 28 - DNA, 56 bp, Artificial Sequence
  Primer
gggggcgtgagctcaccgcctccgccggagccctcagtacctttgttattttcaac

SEQ ID NO: 29 - DNA, 2799 bp, Artificial Sequence
  RH2
atggctagcgctgagacagggacaattacagttcaagatactcaaaaaggcgcaacctataaagcatataaagtttttgatgcagaaatagataatgcaaatgtatctgattcgaataaagatggagcttcttatttaattcctcaaggtaaagaagctgagtataaagcttcaactgattttaattctctttttacgacaactactaatggagggagaacatatgtaactaaaaaagatactgcgtcagcaaatgagattgcgacatgggctaaatctatatcagctaatactacaccagtttccactgttactgagtcaaataatgatggtactgaggttattaatgtttcccaatatggatattattatgtttctagcactgttaataatggtgctgtgattatggttacatctgtaactccaaatgctactattcatgaaaagaatactgatgcgacatggggagatggtggtggaaaaactgtagatcaaaaaacgtactcggttggtgatacagtcaaatatactattacttataagaatgcagtcaattatcatggtacagaaaaagtgtatcaatatgttataaaggatactatgccatctgcttctgtagttgatttgaacgaagggtcttatgaagtaactattactgatggatcagggaatattacaactctaactcaaggttcggaaaaagcaactgggaagtataacctgttagaggaaaataataatttcacgattactattccgtgggcagctaccaatactccaaccggaaatactcaaaatggagctaatgatgactttttttataagggaataaatacaatcacagtcacttatacaggagtattaaagagtggagctaaaccaggttcagctgatttaccagaaaatacaaacattgcgaccatcaaccccaatactagcaatgatgacccaggtcaaaaagtaacagtgagggatggtcaaattactataaaaaaaattgatggttccacaaaagcttcattacaaggtgctatatttgttttaaagaatgctacgggtcaatttctaaactttaacgatacaaataacgttgaatggggcacagaagctaatgcaacagaatatacaacaggagcagatggtataattaccattacaggcttgaaagaaggtacatactatctagttgagaaaaaggctcccttaggttacaatttgttagataactctcagaaggttattttaggagatggagccactgatacgactaattcagataaccttttagttaacccaactgttgaaaataacaaaggtactgagggctccggcggaggcggtgagctcgcagaagtgtcacaagaacgcccagcgaaaacaacagtaaatatctataaattacaagctgatagttataaatcggaaattacttctaatggtggtatcgagaataaagacggcgaagtaatatctaactatgctaaacttggtgacaatgtaaaaggtttgcaaggtgtacagtttaaacgttataaagtcaagacggatatttctgttgatgaattgaaaaaattgacaacagttgaagcagcagatgcaaaagttggaacgattcttgaagaaggtgtcagtctacctcaaaaaactaatgctcaaggtttggtcgtcgatgctctggattcaaaaagtaatgtgagatacttgtatgtagaagatttaaagaattcaccttcaaacattaccaaagcttatgctgtaccgtttgtgttggaattaccagttgctaactctacaggtacaggtttcctttctgaaattaatatttaccctaaaaacgttgtaactgatgaaccaaaaacagataaagatgttaaaaaattaggtcaggacgatgcaggttatacgattggtgaagaattcaaatggttcttgaaatctacaatccctgccaatttaggtgactatgaaaaatttgaaattactgataaatttgcagatggcttgacttataaatctgttggaaaaatcaagattggttcgaaaacactgaatagagatgagcactacactattgatgaaccaacagttgataaccaaaatacattaaaaattacgtttaaaccagagaaatttaaagaaattgctgagctacttaaaggaatgacccttgttaaaaatcaagatgctcttgataaagctactgcaaatacagatgatgcggcatttttggaaattccagttgcatcaactattaatgaaaaagcagttttaggaaaagcaattgaaaatacttttgaacttcaatatgaccatactcctgataaagctgacaatccaaaaccatctaatcctccaagaaaaccagaagttcatactggtgggaaacgatttgtaaagaaagactcaacagaaacacaaacactaggtggtgctgagtttgatttgttggcttctgatgggacagcagtaaaatggacagatgctcttattaaagcgaatactaataaaaactatattgctggagaagctgttactgggcaaccaatcaaattgaaatcacatacagacggtacgtttgagattaaaggtttggcttatgcagttgatgcgaatgcagagggtacagcagtaacttacaaattaaaagaaacaaaagcaccagaaggttatgtaatccctgataaagaaatcgagtttacagtatcacaaacatcttataatacaaaaccaactgacatcacggttgatagtgctgatgcaacacctgatacaattaaaaacaacaaacgtccttcataa

SEQ ID NO: 30 - protein, 947 aa, Artificial Sequence
  RH1 with C-terminal sequence
MASAETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTEGGGGSGGGGSGGGGSELAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSHHHHHH

SEQ ID NO: 31 - DNA, 2826 bp, Artificial Sequence
  RH1 without RBS2
atggctagcgctgagacagggacaattacagttcaagatactcaaaaaggcgcaacctataaagcatataaagtttttgatgcagaaatagataatgcaaatgtatctgattcgaataaagatggagcttcttatttaattcctcaaggtaaagaagctgagtataaagcttcaactgattttaattctctttttacgacaactactaatggagggagaacatatgtaactaaaaaagatactgcgtcagcaaatgagattgcgacatgggctaaatctatatcagctaatactacaccagtttccactgttactgagtcaaataatgatggtactgaggttattaatgtttcccaatatggatattattatgtttctagcactgttaataatggtgctgtgattatggttacatctgtaactccaaatgctactattcatgaaaagaatactgatgcgacatggggagatggtggtggaaaaactgtagatcaaaaaacgtactcggttggtgatacagtcaaatatactattacttataagaatgcagtcaattatcatggtacagaaaaagtgtatcaatatgttataaaggatactatgccatctgcttctgtagttgatttgaacgaagggtcttatgaagtaactattactgatggatcagggaatattacaactctaactcaaggttcggaaaaagcaactgggaagtataacctgttagaggaaaataataatttcacgattactattccgtgggcagctaccaatactccaaccggaaatactcaaaatggagctaatgatgactttttttataagggaataaatacaatcacagtcacttatacaggagtattaaagagtggagctaaaccaggttcagctgatttaccagaaaatacaaacattgcgaccatcaaccccaatactagcaatgatgacccaggtcaaaaagtaacagtgagggatggtcaaattactataaaaaaaattgatggttccacaaaagcttcattacaaggtgctatatttgttttaaagaatgctacgggtcaatttctaaactttaacgatacaaataacgttgaatggggcacagaagctaatgcaacagaatatacaacaggagcagatggtataattaccattacaggcttgaaagaaggtacatactatctagttgagaaaaaggctcccttaggttacaatttgttagataactctcagaaggttattttaggagatggagccactgatacgactaattcagataaccttttagttaacccaactgttgaaaataacaaaggtactgagggtggcggaggctccggcggaggcggttctggtggcggaggcagcgagctcgcagaagtgtcacaagaacgcccagcgaaaacaacagtaaatatctataaattacaagctgatagttataaatcggaaattacttctaatggtggtatcgagaataaagacggcgaagtaatatctaactatgctaaacttggtgacaatgtaaaaggtttgcaaggtgtacagtttaaacgttataaagtcaagacggatatttctgttgatgaattgaaaaaattgacaacagttgaagcagcagatgcaaaagttggaacgattcttgaagaaggtgtcagtctacctcaaaaaactaatgctcaaggtttggtcgtcgatgctctggattcaaaaagtaatgtgagatacttgtatgtagaagatttaaagaattcaccttcaaacattaccaaagcttatgctgtaccgtttgtgttggaattaccagttgctaactctacaggtacaggtttcctttctgaaattaatatttaccctaaaaacgttgtaactgatgaaccaaaaacagataaagatgttaaaaaattaggtcaggacgatgcaggttatacgattggtgaagaattcaaatggttcttgaaatctacaatccctgccaatttaggtgactatgaaaaatttgaaattactgataaatttgcagatggcttgacttataaatctgttggaaaaatcaagattggttcgaaaacactgaatagagatgagcactacactattgatgaaccaacagttgataaccaaaatacattaaaaattacgtttaaaccagagaaatttaaagaaattgctgagctacttaaaggaatgacccttgttaaaaatcaagatgctcttgataaagctactgcaaatacagatgatgcggcatttttggaaattccagttgcatcaactattaatgaaaaagcagttttaggaaaagcaattgaaaatacttttgaacttcaatatgaccatactcctgataaagctgacaatccaaaaccatctaatcctccaagaaaaccagaagttcatactggtgggaaacgatttgtaaagaaagactcaacagaaacacaaacactaggtggtgctgagtttgatttgttggcttctgatgggacagcagtaaaatggacagatgctcttattaaagcgaatactaataaaaactatattgctggagaagctgttactgggcaaccaatcaaattgaaatcacatacagacggtacgtttgagattaaaggtttggcttatgcagttgatgcgaatgcagagggtacagcagtaacttacaaattaaaagaaacaaaagcaccagaaggttatgtaatccctgataaagaaatcgagtttacagtatcacaaacatcttataatacaaaaccaactgacatcacggttgatagtgctgatgcaacacctgatacaattaaaaacaacaaacgtccttcataa

SEQ ID NO: 32 - protein, 438 aa, Artificial Sequence
  SpbI with point mutation
AETGTITVQDTKKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTE

SEQ ID NO: 33 - DNA, 2826 bp, Artificial Sequence
  RH1′
atggctagcgctgagacagggacaattacagttcaagatactaaaaaaggcgcaacctataaagcatataaagtttttgatgcagaaatagataatgcaaatgtatctgattcgaataaagatggagcttcttatttaattcctcaaggtaaagaagctgagtataaagcttcaactgattttaattctctttttacgacaactactaatggagggagaacatatgtaactaaaaaagatactgcgtcagcaaatgagattgcgacatgggctaaatctatatcagctaatactacaccagtttccactgttactgagtcaaataatgatggtactgaggttattaatgtttcccaatatggatattattatgtttctagcactgttaataatggtgctgtgattatggttacatctgtaactccaaatgctactattcatgaaaagaatactgatgcgacatggggagatggtggtggaaaaactgtagatcaaaaaacgtactcggttggtgatacagtcaaatatactattacttataagaatgcagtcaattatcatggtacagaaaaagtgtatcaatatgttataaaggatactatgccatctgcttctgtagttgatttgaacgaagggtcttatgaagtaactattactgatggatcagggaatattacaactctaactcaaggttcggaaaaagcaactgggaagtataacctgttagaggaaaataataatttcacgattactattccgtgggcagctaccaatactccaaccggaaatactcaaaatggagctaatgatgactttttttataagggaataaatacaatcacagtcacttatacaggagtattaaagagtggagctaaaccaggttcagctgatttaccagaaaatacaaacattgcgaccatcaaccccaatactagcaatgatgacccaggtcaaaaagtaacagtgagggatggtcaaattactataaaaaaaattgatggttccacaaaagcttcattacaaggtgctatatttgttttaaagaatgctacgggtcaatttctaaactttaacgatacaaataacgttgaatggggcacagaagctaatgcaacagaatatacaacaggagcagatggtataattaccattacaggcttgaaagaaggtacatactatctagttgagaaaaaggctcccttaggttacaatttgttagataactctcagaaggttattttaggagatggagccactgatacgactaattcagataaccttttagttaacccaactgttgaaaataacaaaggtactgagggtggcggaggctccggcggaggcggttctggtggcggaggcagcgagctcgcagaagtgtcacaagaacgcccagcgaaaacaacagtaaatatctataaattacaagctgatagttataaatcggaaattacttctaatggtggtatcgagaataaagacggcgaagtaatatctaactatgctaaacttggtgacaatgtaaaaggtttgcaaggtgtacagtttaaacgttataaagtcaagacggatatttctgttgatgaattgaaaaaattgacaacagttgaagcagcagatgcaaaagttggaacgattcttgaagaaggtgtcagtctacctcaaaaaactaatgctcaaggtttggtcgtcgatgctctggattcaaaaagtaatgtgagatacttgtatgtagaagatttaaagaattcaccttcaaacattaccaaagcttatgctgtaccgtttgtgttggaattaccagttgctaactctacaggtacaggtttcctttctgaaattaatatttaccctaaaaacgttgtaactgatgaaccaaaaacagataaagatgttaaaaaattaggtcaggacgatgcaggttatacgattggtgaagaattcaaatggttcttgaaatctacaatccctgccaatttaggtgactatgaaaaatttgaaattactgataaatttgcagatggcttgacttataaatctgttggaaaaatcaagattggttcgaaaacactgaatagagatgagcactacactattgatgaaccaacagttgataaccaaaatacattaaaaattacgtttaaaccagagaaatttaaagaaattgctgagctacttaaaggaatgacccttgttaaaaatcaagatgctcttgataaagctactgcaaatacagatgatgcggcatttttggaaattccagttgcatcaactattaatgaaaaagcagttttaggaaaagcaattgaaaatacttttgaacttcaatatgaccatactcctgataaagctgacaatccaaaaccatctaatcctccaagaaaaccagaagttcatactggtgggaaacgatttgtaaagaaagactcaacagaaacacaaacactaggtggtgctgagtttgatttgttggcttctgatgggacagcagtaaaatggacagatgctcttattaaagcgaatactaataaaaactatattgctggagaagctgttactgggcaaccaatcaaattgaaatcacatacagacggtacgtttgagattaaaggtttggcttatgcagttgatgcgaatgcagagggtacagcagtaacttacaaattaaaagaaacaaaagcaccagaaggttatgtaatccctgataaagaaatcgagtttacagtatcacaaacatcttataatacaaaaccaactgacatcacggttgatagtgctgatgcaacacctgatacaattaaaaacaacaaacgtccttcataa

SEQ ID NO: 34 - protein, 941 aa, Artificial Sequence
  RH1′
MASAETGTITVQDTKKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTEGGGGSGGGGSGGGGSELAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 35 - DNA, 2799 bp, Artificial Sequence
  RH2′
atggctagcgctgagacagggacaattacagttcaagatactaaaaaaggcgcaacctataaagcatataaagtttttgatgcagaaatagataatgcaaatgtatctgattcgaataaagatggagcttcttatttaattcctcaaggtaaagaagctgagtataaagcttcaactgattttaattctctttttacgacaactactaatggagggagaacatatgtaactaaaaaagatactgcgtcagcaaatgagattgcgacatgggctaaatctatatcagctaatactacaccagtttccactgttactgagtcaaataatgatggtactgaggttattaatgtttcccaatatggatattattatgtttctagcactgttaataatggtgctgtgattatggttacatctgtaactccaaatgctactattcatgaaaagaatactgatgcgacatggggagatggtggtggaaaaactgtagatcaaaaaacgtactcggttggtgatacagtcaaatatactattacttataagaatgcagtcaattatcatggtacagaaaaagtgtatcaatatgttataaaggatactatgccatctgcttctgtagttgatttgaacgaagggtcttatgaagtaactattactgatggatcagggaatattacaactctaactcaaggttcggaaaaagcaactgggaagtataacctgttagaggaaaataataatttcacgattactattccgtgggcagctaccaatactccaaccggaaatactcaaaatggagctaatgatgactttttttataagggaataaatacaatcacagtcacttatacaggagtattaaagagtggagctaaaccaggttcagctgatttaccagaaaatacaaacattgcgaccatcaaccccaatactagcaatgatgacccaggtcaaaaagtaacagtgagggatggtcaaattactataaaaaaaattgatggttccacaaaagcttcattacaaggtgctatatttgttttaaagaatgctacgggtcaatttctaaactttaacgatacaaataacgttgaatggggcacagaagctaatgcaacagaatatacaacaggagcagatggtataattaccattacaggcttgaaagaaggtacatactatctagttgagaaaaaggctcccttaggttacaatttgttagataactctcagaaggttattttaggagatggagccactgatacgactaattcagataaccttttagttaacccaactgttgaaaataacaaaggtactgagggctccggcggaggcggtgagctcgcagaagtgtcacaagaacgcccagcgaaaacaacagtaaatatctataaattacaagctgatagttataaatcggaaattacttctaatggtggtatcgagaataaagacggcgaagtaatatctaactatgctaaacttggtgacaatgtaaaaggtttgcaaggtgtacagtttaaacgttataaagtcaagacggatatttctgttgatgaattgaaaaaattgacaacagttgaagcagcagatgcaaaagttggaacgattcttgaagaaggtgtcagtctacctcaaaaaactaatgctcaaggtttggtcgtcgatgctctggattcaaaaagtaatgtgagatacttgtatgtagaagatttaaagaattcaccttcaaacattaccaaagcttatgctgtaccgtttgtgttggaattaccagttgctaactctacaggtacaggtttcctttctgaaattaatatttaccctaaaaacgttgtaactgatgaaccaaaaacagataaagatgttaaaaaattaggtcaggacgatgcaggttatacgattggtgaagaattcaaatggttcttgaaatctacaatccctgccaatttaggtgactatgaaaaatttgaaattactgataaatttgcagatggcttgacttataaatctgttggaaaaatcaagattggttcgaaaacactgaatagagatgagcactacactattgatgaaccaacagttgataaccaaaatacattaaaaattacgtttaaaccagagaaatttaaagaaattgctgagctacttaaaggaatgacccttgttaaaaatcaagatgctcttgataaagctactgcaaatacagatgatgcggcatttttggaaattccagttgcatcaactattaatgaaaaagcagttttaggaaaagcaattgaaaatacttttgaacttcaatatgaccatactcctgataaagctgacaatccaaaaccatctaatcctccaagaaaaccagaagttcatactggtgggaaacgatttgtaaagaaagactcaacagaaacacaaacactaggtggtgctgagtttgatttgttggcttctgatgggacagcagtaaaatggacagatgctcttattaaagcgaatactaataaaaactatattgctggagaagctgttactgggcaaccaatcaaattgaaatcacatacagacggtacgtttgagattaaaggtttggcttatgcagttgatgcgaatgcagagggtacagcagtaacttacaaattaaaagaaacaaaagcaccagaaggttatgtaatccctgataaagaaatcgagtttacagtatcacaaacatcttataatacaaaaccaactgacatcacggttgatagtgctgatgcaacacctgatacaattaaaaacaacaaacgtccttcataa

SEQ ID NO: 36 - protein, 931 aa, Artificial Sequence
  RH2′
MASAETGTITVQDTKKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTEGSGGGGELAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 37 - DNA, 2802 bp, Artificial Sequence
  Codon-optimised RH2
atggctagcgcagaaaccggcaccattaccgttcaggatacccagaaaggtgcaacctataaagcgtataaagtgtttgatgccgaaattgataatgccaatgtgagcgatagcaataaagatggtgccagctatctgattccgcagggtaaagaagcagaatataaagccagcaccgattttaatagcctgtttaccaccacaaccaatggtggtcgtacctatgtgaccaaaaaagataccgcaagcgcaaatgaaattgcaacatgggcaaaaagcattagcgcaaataccacaccggttagcaccgttaccgaaagcaataatgatggcaccgaagtgattaatgttagccagtatggctattattatgttagcagcaccgttaataatggtgccgttattatggttaccagcgttaccccgaatgcaaccattcatgaaaaaaacaccgatgcaacctggggtgatggcggtggtaaaaccgttgatcagaaaacctattctgttggcgataccgtgaaatacaccattacctataaaaatgccgtgaattatcatggcaccgaaaaagtgtatcagtatgtgattaaagataccatgccgagcgcaagcgttgttgatctgaatgaaggcagctatgaagttaccattaccgatggtagcggcaatattaccaccctgacccagggtagcgaaaaagcaaccggcaaatataatctgctggaagaaaataataattttaccattaccattccgtgggcagcaaccaataccccgaccggtaatacccagaatggcgcaaatgatgattttttttataaaggcattaataccattaccgtgacctataccggtgttctgaaaagcggtgcaaaaccgggtagcgcagatctgccggaaaataccaatattgccaccattaatccgaataccagcaatgatgatccgggtcagaaagttaccgttcgtgatggtcagattaccattaaaaaaattgatggcagcaccaaagcaagcctgcagggtgcaatttttgttctgaaaaatgccaccggtcagtttctgaattttaatgataccaataatgtggaatggggcaccgaagcaaatgcaaccgaatataccaccggtgcagatggtattattaccattacaggcctgaaagaaggcacctattacctggtggaaaaaaaagcaccgctgggttataacctgctggataacagccagaaagttattctgggtgatggtgccaccgataccaccaatagcgacaacctgctggtgaatccgaccgtggaaaataataaaggcaccgaaggtagcggtggtggtggtgaactggccgaagttagccaggaacgtccggctaaaaccaccgtgaatatttataaactgcaggccgatagctataaaagcgaaattaccagcaatggtggcattgaaaataaagatggcgaagtgattagcaattatgccaaactgggcgataatgttaaaggtctgcagggtgttcagtttaaacgctataaagtgaaaaccgatattagcgtggatgaactgaaaaaactgaccaccgttgaagcagcagatgcaaaagttggcaccattctggaagaaggtgttagcctgccgcagaaaaccaatgcacagggtctggttgttgatgcactggatagcaaaagcaatgtgcgctatctgtatgtggaagatctgaaaaatagcccgagcaatattactaaagcatatgccgttccgtttgttctggaactgccggttgcaaatagcaccggcaccggttttctgagcgaaatcaatatttatccgaaaaatgtggttaccgatgaaccgaaaaccgataaagatgtgaaaaaactgggtcaggatgatgcaggttataccattggcgaagaatttaaatggtttctgaaaagcaccattccggcaaatctgggcgattatgaaaaatttgaaattaccgataaatttgccgatggcctgacctataaaagcgtgggcaaaattaaaattggcagcaaaaccctgaatcgtgatgaacattataccattgatgaaccgaccgttgataatcagaataccctgaaaattacctttaaaccggaaaaatttaaagaaattgccgaactgctgaaaggtatgaccctggtgaaaaatcaggatgccctggataaagcaaccgccaataccgatgatgcagcctttctggaaattccggttgccagcaccattaatgaaaaagccgttctgggtaaagcaattgaaaatacctttgaactgcagtatgatcatacaccggataaagccgataatccgaaaccgtctaatcctccgcgtaaaccggaagttcataccggtggtaaacgctttgtgaaaaaagatagcaccgaaacccagaccctgggtggtgcagaatttgatctgctggcctctgatggcaccgcagttaaatggaccgatgcactgattaaagccaataccaataagaattacattgccggtgaagcagttaccggtcagccgattaaactgaaaagccataccgatggcacctttgaaattaaaggtctggcctatgcagttgatgcaaatgcagaaggtacagccgttacctataaactgaaagaaaccaaagcaccggaaggttatgttattccggataaagaaattgaatttaccgttagccagaccagctataacaccaaaccgaccgatattaccgttgattctgcagatgcaacaccggatacgattaaaaataataaacgtccgagctaataa

SEQ ID NO: 38 - DNA, 2802 bp, Artificial Sequence
  Codon-optimised RH2′
atggctagcgcagaaaccggcaccattaccgttcaggataccaaaaaaggtgcaacctataaagcgtataaagtgtttgatgccgaaattgataatgccaatgtgagcgatagcaataaagatggtgccagctatctgattccgcagggtaaagaagcagaatataaagccagcaccgattttaatagcctgtttaccaccacaaccaatggtggtcgtacctatgtgaccaaaaaagataccgcaagcgcaaatgaaattgcaacatgggcaaaaagcattagcgcaaataccacaccggttagcaccgttaccgaaagcaataatgatggcaccgaagtgattaatgttagccagtatggctattattatgttagcagcaccgttaataatggtgccgttattatggttaccagcgttaccccgaatgcaaccattcatgaaaaaaacaccgatgcaacctggggtgatggcggtggtaaaaccgttgatcagaaaacctattctgttggcgataccgtgaaatacaccattacctataaaaatgccgtgaattatcatggcaccgaaaaagtgtatcagtatgtgattaaagataccatgccgagcgcaagcgttgttgatctgaatgaaggcagctatgaagttaccattaccgatggtagcggcaatattaccaccctgacccagggtagcgaaaaagcaaccggcaaatataatctgctggaagaaaataataattttaccattaccattccgtgggcagcaaccaataccccgaccggtaatacccagaatggcgcaaatgatgattttttttataaaggcattaataccattaccgtgacctataccggtgttctgaaaagcggtgcaaaaccgggtagcgcagatctgccggaaaataccaatattgccaccattaatccgaataccagcaatgatgatccgggtcagaaagttaccgttcgtgatggtcagattaccattaaaaaaattgatggcagcaccaaagcaagcctgcagggtgcaatttttgttctgaaaaatgccaccggtcagtttctgaattttaatgataccaataatgtggaatggggcaccgaagcaaatgcaaccgaatataccaccggtgcagatggtattattaccattacaggcctgaaagaaggcacctattacctggtggaaaaaaaagcaccgctgggttataacctgctggataacagccagaaagttattctgggtgatggtgccaccgataccaccaatagcgacaacctgctggtgaatccgaccgtggaaaataataaaggcaccgaaggtagcggtggtggtggtgaactggccgaagttagccaggaacgtccggctaaaaccaccgtgaatatttataaactgcaggccgatagctataaaagcgaaattaccagcaatggtggcattgaaaataaagatggcgaagtgattagcaattatgccaaactgggcgataatgttaaaggtctgcagggtgttcagtttaaacgctataaagtgaaaaccgatattagcgtggatgaactgaaaaaactgaccaccgttgaagcagcagatgcaaaagttggcaccattctggaagaaggtgttagcctgccgcagaaaaccaatgcacagggtctggttgttgatgcactggatagcaaaagcaatgtgcgctatctgtatgtggaagatctgaaaaatagcccgagcaatattactaaagcatatgccgttccgtttgttctggaactgccggttgcaaatagcaccggcaccggttttctgagcgaaatcaatatttatccgaaaaatgtggttaccgatgaaccgaaaaccgataaagatgtgaaaaaactgggtcaggatgatgcaggttataccattggcgaagaatttaaatggtttctgaaaagcaccattccggcaaatctgggcgattatgaaaaatttgaaattaccgataaatttgccgatggcctgacctataaaagcgtgggcaaaattaaaattggcagcaaaaccctgaatcgtgatgaacattataccattgatgaaccgaccgttgataatcagaataccctgaaaattacctttaaaccggaaaaatttaaagaaattgccgaactgctgaaaggtatgaccctggtgaaaaatcaggatgccctggataaagcaaccgccaataccgatgatgcagcctttctggaaattccggttgccagcaccattaatgaaaaagccgttctgggtaaagcaattgaaaatacctttgaactgcagtatgatcatacaccggataaagccgataatccgaaaccgtctaatcctccgcgtaaaccggaagttcataccggtggtaaacgctttgtgaaaaaagatagcaccgaaacccagaccctgggtggtgcagaatttgatctgctggcctctgatggcaccgcagttaaatggaccgatgcactgattaaagccaataccaataagaattacattgccggtgaagcagttaccggtcagccgattaaactgaaaagccataccgatggcacctttgaaattaaaggtctggcctatgcagttgatgcaaatgcagaaggtacagccgttacctataaactgaaagaaaccaaagcaccggaaggttatgttattccggataaagaaattgaatttaccgttagccagaccagctataacaccaaaccgaccgatattaccgttgattctgcagatgcaacaccggatacgattaaaaataataaacgtccgagctaataa

(19) United States
(12) Patent Application Publication
(10) Pub. No.: US 2011/0052620 A1
(43) Pub. Date: Mar. 3, 2011

(54) HYBRID POLYPEPTIDES COMPRISING GBS-80 AND SPB1 PROTEINS OF STREPTOCOCCUS

(75) Inventors: Immaculada Margarit Y Ros, Siena (IT); Guido Grandi, Segrate (IT); John L. Telford, Monteriggioni (IT); Domenico Maione, Siena (IT)

(73) Assignee: Novartis Vaccines and Diagnostics SRL, Siena (IT)

(21) Appl. No.: 12/866,845

(22) PCT Filed: Feb. 11, 2009
(86) PCT No.: PCT/GB09/00384

§ 371 Date: Aug. 9, 2010

(30) Foreign Application Priority Data
Feb. 11, 2008 (GB) 0802503.3

(51) Int. Cl. A61K 39/09; C07K 14/315; C07H 21/04; A61P 31/04

(52) U.S. Cl. 424/190.1; 530/350; 536/23.1

(57) ABSTRACT

GBS antigens GBS-80 and Spb1 are expressed in a single polypeptide chain, preferably with Spb1 upstream of GBS-80. Thus the invention provides a polypeptide comprising an amino acid sequence NH₂—W—X-L-Y—Z—CO₂H wherein: X is a Spb 1 sequence; L is an optional linker; Y is a GBS-80 sequence; W is an optional N-terminal sequence; and Z is an optional C-terminal sequence.

This application claims the benefit of GB0802503.3 filed on 11 Feb. 2008, the complete contents of which are incorporated herein by reference.

### TECHNICAL FIELD

This invention is in the field of immunising against Streptococcus agalactiae (GBS).

### BACKGROUND ART

References 1 and 2 disclose how multistrain genome analysis has been used to identify antigens for including a universal GBS vaccine. The best four candidates identified by this procedure were referred to as GBS-67, GBS-80, GBS-104 and GBS-322.

There remains a need to identify further and improved antigens for vaccinating against GBS.

### DISCLOSURE OF THE INVENTION

The invention concerns two GBS antigens: GBS-80 and Spb1. Both of these proteins are pilus backbone antigens, but they are present on different pilus islands (GBS-80 is found in Pilus Island I, and Spb1 in Pilus island 2b). According to the invention these two pilus antigens are expressed in a single polypeptide chain, preferably with Spb1 upstream of GBS-80. Expression of both antigens in a single polypeptide offers two main advantages: first, the stability of Spb1 and/or GBS-80 can be enhanced; second, commercial manufacture is simplified as only one expression and purification needs be employed in order to produce both of the antigens.

Thus the invention provides a polypeptide comprising an amino acid sequence X-L-Y, wherein: X is a Spb1 sequence; L is an optional linker; and Y is a GBS-80 sequence.

The polypeptide may optionally comprise sequence upstream of X and/or downstream of Y, in which case the polypeptide has amino acid sequence NH₂—W—X-L-Y—Z—CO₂H, wherein: X, L & Y are as defined above; W is an optional N-terminal sequence; and Z is an optional C-terminal sequence.

In a separate aspect of the invention, two translation initiation sites (RBS) have been identified in the natural Spb1 transcript, which leads in a recombinant host to the expression of two polypeptide products. Mutation of the second RBS gives improved yields of the longer polypeptide.

Thus the invention provides a nucleic acid encoding a Spb1 sequence, wherein the Spb1 sequence includes a N-terminus methionine start codon and an internal methionine codon, and wherein the nucleic acid does not include a GGAG 4-mer nucleotide sequence in the 15 nucleotides upstream of the internal methionine codon.

X: Spb1 Sequence

Polypeptides of the invention include a X moiety which is a Spb1 sequence. This Spb1 sequence will, when administered to a subject, elicit an antibody response comprising antibodies that bind to wild-type Spb1 protein e.g. to the S. agalactiae protein having amino acid sequence SEQ ID NO: 1 (the full-length wild-type sequence from strain COH1).

The Spb1 sequence may comprise an amino acid sequence having at least a % identity to SEQ ID NO: 2. The value of a may be selected from 50, 60, 70, 80, 85, 90, 95, 96, 97, 98, 99 or more. The Spb1 sequence may comprise SEQ ID NO: 2.

The Spb1 sequence may comprise a fragment of SEQ ID NO: 1 and/or of SEQ ID NO: 2. The fragment will usually include at least b amino acids of SEQ ID NO: 1/2, wherein b is selected from 5, 6, 7, 8, 9, 10, 11, 12, 13, 14, 15, 16, 17, 18, 19, 20, 22, 24, 26, 28, 30, 40, 50, 60, 70, 80, 90, 100, 110, 120, 130, 140, 150, 175, 200 or more. The fragment will usually include at least one T-cell or, preferably, a B-cell epitope of SEQ ID NO: 1/2. T- and B-cell epitopes can be identified empirically (e.g. using PEPSCAN [3,4] or similar methods), or they can be predicted (e.g. using the Jameson-Wolf antigenic index [5], matrix-based approaches [6], TEPITOPE [7], neural networks [8], OptiMer & EpiMer [9,10], ADEPT [11], Tsites [12], hydrophilicity [13], antigenic index [14] or the methods disclosed in reference 15, etc.). SEQ ID NO: 2 is itself a fragment of SEQ ID NO: 1.

The Spb1 sequence may comprise an amino acid sequence that has both at least a % identity to SEQ ID NO: 2 and comprises a fragment of SEQ ID NO: 2, as defined above.

The X moiety will usually be at least c amino acids long, where c is selected from 50, 60, 70, 80, 90, 100, 120, 140, 160, 180, 200, 220, 240, 260, 280, 300, 320, 340, 360, 380, 400 or more.

The X moiety will usually be no longer than d amino acids long, where d is selected from 500, 480, 460, 440, 420, 400, 380, 360, 340, 320, 300, 280, 260, 240, 220, 200 or less.

The X moiety will usually be between 300-500 amino acids long e.g. 350-480, 400-460, 430-450.

The wild-type SpbI sequence from serotype III strain COH1 is SEQ ID NO: 1 herein:

MKKKMIQSLLVASLAFGMAVSPVTPIAFAAETGTITVQDTQKGATYKAYK
VFDAEIDNANVSDSNKDGASYLIPQGKEAEYKASTDFNSLFTTTTNGGRT
YVTKKDTASANEIATWAKSISANTTPVSTVTESNNDGTEVINVSQYGYYY
VSSTVNNGAVIMVTSVTPNATIHEKNTDATWGDGGGKTVDQKTYSVGDTV
KYTITYKNAVNYHGTEKVYQYVIKDTMPSASVVDLNEGSYEVTITDGSGN
ITTLTQGSEKATGKYNLLEENNNFTITIPWAATNTPTGNTQNGANDDFFY
KGINTITVTYTGVLKSGAKPGSADLPENTNIATINPNTSNDDPGQKVTVR
DGQITIKKIDGSTKASLQGAIFVLKNATGQFLNFNDTNNVEWGTEANATE
YTTGADGIITITGLKEGTYYLVEKKAPLGYNLLDNSQKVILGDGATDTTN
SDNLLVNPTVENNKGTELPSTGGIGTTIFYIIGAILVIGAGIVLVARRRL
RS

Wild-type SpbI contains a N-terminal leader or signal sequence region which is indicated by the underlined sequence above (aa 1-29). The wild-type sequence contains an amino acid motif indicative of a cell wall anchor (LPSTG, underlined). In some recombinant host cell systems, it may be preferable to remove this motif to facilitate secretion of a recombinant polypeptide from the cell. Alternatively, it may be preferable to use the cell wall anchor motif to anchor the recombinantly expressed polypeptide to the cell wall. The extracellular domain of the expressed polypeptide may be cleaved during purification or the recombinant polypeptide may be left attached to either inactivated host cells or cell membranes in the final composition. An E box containing a conserved glutamic residue has also been identified in SpbI (underlined), with a conserved glutamic acid at residue 423. The E box motif may be important for the formation of oligomeric pilus-like structures, and so useful fragments of SpbI may include the conserved glutamic acid residue. A mutant of SpbI has been identified in which the glutamine (Q) at position 41 of the wild-type sequence (bold above) is substituted for a lysine (K), as a result of a mutation of a codon in the encoding nucleotide sequence from CAA to AAA. This substitution may be present in SpbI sequences and SpbI fragments (e.g. SEQ ID NO:32).

The wild-type Spb1 sequence includes an internal methionine codon (Met-162) that has an upstream 12-mer TAATGGAGCTGT sequence (SEQ ID NO: 12) that includes the core sequence (underlined) of a Shine-Dalgarno sequence. This Shine-Dalgarno sequence has been found to initiate translation of a truncated Spb1 sequence. To prevent translation initiation at this site the Shine-Dalgarno sequence can be disrupted in a Spb1-coding sequence used for expression according to the invention. Although any suitable nucleotide can be mutated to prevent ribosome binding, the sequence includes a GGA glycine codon that is both part of the Shine-Dalgarno core and in-frame with the internal methionine codon. The third base in this codon can be mutated to C, G or T without changing the encoded glycine, thereby avoiding any change in Spb1 sequence.

Y: GBS-80 Sequence

Polypeptides of the invention include a Y moiety which is a GBS-80 sequence. This GBS-80 sequence will, when administered to a subject, elicit an antibody response comprising antibodies that bind to wild-type GBS-80 protein e.g. to the S. agalactiae protein having amino acid sequence SEQ ID NO: 3 (the full-length wild-type sequence from strain 2603V/R).

The GBS-80 sequence may comprise an amino acid sequence having at least e % identity to SEQ ID NO: 4. The value of e may be selected from 50, 60, 70, 80, 85, 90, 95, 96, 97, 98, 99 or more. The GBS-80 sequence may comprise SEQ ID NO: 4.

The GBS-80 sequence may comprise a fragment of SEQ ID NO: 3 or of SEQ ID NO: 4. The fragment will usually include at least f amino acids of SEQ ID NO: 3/4, wherein f is selected from 5, 6, 7, 8, 9, 10, 11, 12, 13, 14, 15, 16, 17, 18, 19, 20, 22, 24, 26, 28, 30, 40, 50, 60, 70, 80, 90, 100, 110, 120, 130, 140, 150, 175, 200 or more. The fragment will usually include at least one T-cell or, preferably, B-cell epitope of SEQ ID NO: 3/4. SEQ ID NO: 4 is itself a fragment of SEQ ID NO: 3.

The GBS-80 sequence may comprise an amino acid sequence that has both at least e % identity to SEQ ID NO: 4 and comprises a fragment of SEQ ID NO: 4, as defined above.

The Y moiety will usually be at least g amino acids long, where g is selected from 50, 60, 70, 80, 90, 100, 120, 140, 160, 180, 200, 220, 240, 260, 280, 300, 320, 340, 360, 380, 400, 420, 440, 460, 480, 500, 520, 540, 560, 580, 600 or more.

The Y moiety will usually be no longer than h amino acids long, where h is selected from 600, 580, 560, 540, 520, 500, 480, 460, 440, 420, 400, 380, 360, 340, 320, 300, 280, 260, 240, 220, 200 or less.

The Y moiety will usually be between 350-550 amino acids long e.g. 400-520, 450-500, 470-490.

The wild-type GBS-80 sequence from serotype V isolated strain 2603 V/R is given in reference 16 as SEQ ID NOs 8779 & 8780. The amino acid sequence is SEQ ID NO: 3 herein:

MKLSKKLLFSAAVLTMVAGSTVEPVAQFATGMSIVRAAEVSQERPAKTTV
NIYKLQADSYKSEITSNGGIENKDGEVISNYAKLGDNVKGLQGVQFKRYK
VKTDISVDELKKLTTVEAADAKVGTILEEGVSLPQKTNAQGLVVDALDSK
SNVRYLYVEDLKNSPSNITKAYAVPFVLELPVANSTGTGFLSEINIYPKN
VVTDEPKTDKDVKKLGQDDAGYTIGEEFKWFLKSTIPANLGDYEKFEITD
KFADGLTYKSVGKIKIGSKTLNRDEHYTIDEPTVDNQNTLKITFKPEKFK
EIAELLKGMTLVKNQDALDKATANTDDAAFLEIPVASTINEKAVLGKAIE
NTFELQYDHTPDKADNPKPSNPPRKPEVHTGGKRFVKKDSTETQTLGGAE
FDLLASDGTAVKWTDALIKANTNKNYIAGEAVTGQPIKLKSHTDGTFEIK
GLAYAVDANAEGTAVTYKLKETKAPEGYVIPDKEIEFTVSQTSYNTKPTD
ITVDSADATPDTIKNNKRPSIPNTGGIGTAIFVAIGAAVMAFAVKGMKRR
TKDN

Wild-type GBS-80 contains a N-terminal leader or signal sequence region which is indicated by the underlined sequence above. One or more amino acids from the leader or signal sequence region of GBS80 can be removed, as in SEQ ID NO: 6 of reference 17. The wild-type sequence also contains a C-terminal transmembrane region which is indicated by the underlined sequence near the end of SEQ ID NO: 3 above. One or more amino acids from the transmembrane region and/or a cytoplasmic region may be removed. An example of such a fragment is SEQ ID NO:7 in reference 17. Wild-type GBS-80 contains an amino acid motif indicative of a cell wall anchor, shown in italics above. In some recombinant host cell systems it may be useful to remove this motif to facilitate secretion of a recombinant GBS80 polypeptide from the host cell. Thus the transmembrane and/or cytoplasmic regions and the cell wall anchor motif may be removed from GBS-80, as shown in SEQ ID NO: 8 of reference 17. Alternatively, in some recombinant host cell systems it may be useful to use the cell wall anchor motif to anchor the recombinantly expressed polypeptide to the cell wall. The extracellular domain of the expressed polypeptide may be cleaved during purification or the recombinant polypeptide may be left attached to either inactivated host cells or cell membranes in the final composition. See SEQ ID NO: 9 of reference 17. A particularly immunogenic fragment of wild-type GBS-80 is located towards the N-terminus of the polypeptide, and is SEQ ID NO: 10 of reference 17 (SEQ ID NO: 5 herein).

L: Linker

Polypeptides of the invention optionally include a L moiety to link the X and Y moieties. The L moiety is typically a short amino acid sequence e.g. in the range of 2-40 amino acids e.g. consisting of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids.

Linkers will usually contain at least one glycine residue, thereby facilitating structural flexibility. The linker may contain, for example, 1, 2, 3, 4, 5, 6, 7, 8, 9, 10 or more glycine residues. The glycines may be arranged to include at least two consecutive glycines in a Gly-Gly dipeptide sequence, or a longer oligo-Gly sequence i.e. Glyₙ where n=2, 3, 4, 5, 6, 7, 8, 9, 10 or more (e.g. SEQ ID NO: 21).

Linkers may be encoded by codons found in the recognition sequences of restriction enzymes. For example, a 6-mer sequence that is the target of a particular restriction enzyme can code for a dipeptide. Thus the recognition sequence for BamHI (GGATCC) encodes Gly-Ser, and so a linker may include a Gly-Ser dipeptide sequence. Such sequences facilitate cloning and manipulation.

Useful linker sequences include SEQ ID Nos 6, 7, 21 and 25.

However, preferred linkers do not include a sequence that shares 10 or more contiguous amino acids in common with a human polypeptide sequence. For instance, one glycine-rich linker sequence that can be used with the invention is the 14mer SEQ ID NO: 6, but this 14 mer is also found in a human RNA binding protein (gi: 8051631) and so it is preferably avoided within the L moiety.

W: N-Terminal Sequence

The X moiety may be at the N-terminus of a polypeptide of the invention, but it is also possible to have amino acids upstream of X. These optional amino acids form a W moiety.

The W moiety is typically a short amino acid sequence e.g. in the range of 2-40 amino acids e.g. consisting of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids.

Examples of W moieties are leader sequences to direct protein trafficking, or comprise short peptide sequences which facilitate cloning or purification (e.g. histidine tags i.e. His, where n=3, 4, 5, 6, 7, 8, 9, 10 or more). Other suitable N-terminal amino acid sequences will be apparent to those skilled in the art.

In a nascent polypeptide the W moiety can provide the polypeptide's N-terminal methionine (formyl-methionine, fMet, in bacteria). One or more amino acids may be cleaved from the N-terminus of a nascent W moiety, however, such that the W moiety in a polypeptide of the invention does not necessarily include a N-terminal methionine.

Useful W moieties include SEQ ID NO 8.

Z: C-Terminal Sequence

The Y moiety may be at the C-terminus of a polypeptide of the invention, but it is also possible to have amino acids downstream of Y. These optional amino acids form a Z moiety.

The Z moiety is typically a short amino acid sequence e.g. in the range of 2-40 amino acids e.g. consisting of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids.

Examples of Z moieties include sequences to direct protein trafficking, short peptide sequences which facilitate cloning or purification (e.g. comprising histidine tags i.e. His, where n=3, 4, 5, 6, 7, 8, 9, 10 or more), or sequences which enhance protein stability. Other suitable C-terminal amino acid sequences will be apparent to those skilled in the art, such as a glutathione-S-transferase, thioredoxin, 14 kDa fragment of S. aureus protein A, a biotinylated peptide, a maltose-binding protein, an enterokinase flag, etc. One useful Z moiety comprises SEQ ID NO 9.

Useful Combinations

Of the various X, Y and L moieties, useful combinations include, but are not limited to:

| SEQ ID | W | X | L | Y | Z |
|---|---|---|---|---|---|
| 10 | 8 | 2 | 7 | 4 | — |
| 30 | 8 | 2 | 7 | 4 | 9 |
| 26 | 8 | 2 | 25 | 4 | — |
| 34 | 8 | 32 | 7 | 4 | — |
| 36 | 8 | 32 | 25 | 4 | — |

The invention provides a polypeptide comprising an amino acid sequence having at least i % sequence identity to SEQ ID NO: 10. The value of i may be selected from 50, 60, 70, 80, 85, 90, 95, 96, 97, 98, 99 or more. The polypeptide may comprise SEQ ID NO: 10.

The invention provides a polypeptide comprising an amino acid sequence having at least i % sequence identity to SEQ ID NO: 34. The value of i may be selected from 50, 60, 70, 80, 85, 90, 95, 96, 97, 98, 99 or more. The polypeptide may comprise SEQ ID NO: 34.

The invention provides a polypeptide comprising an amino acid sequence having at least i % sequence identity to SEQ ID NO: 26. The value of i may be selected from 50, 60, 70, 80, 85, 90, 95, 96, 97, 98, 99 or more. The polypeptide may comprise SEQ ID NO: 26.

The invention provides a polypeptide comprising an amino acid sequence having at least i % sequence identity to SEQ ID NO: 36. The value of i may be selected from 50, 60, 70, 80, 85, 90, 95, 96, 97, 98, 99 or more. The polypeptide may comprise SEQ ID NO: 36.

A polypeptide used with the invention may comprise an amino acid sequence that:
(a) is identical (i.e. 100% identical) to SEQ ID NO: 10 or 26 or 34 or 36;
(b) shares sequence identity SEQ ID NO: 10 or 26 or 34 or 36;
(c) has 1, 2, 3, 4, 5, 6, 7, 8, 9 or 10 (or more) single amino acid alterations (deletions, insertions, substitutions), which may be at separate locations or may be contiguous, as compared to the sequences of (a) or (b); and
(d) when aligned SEQ ID 10 or 26 or 34 or 36 using a pairwise alignment algorithm, each moving window of x amino acids from N-terminus to C-terminus (such that for an alignment that extends to p amino acids, where p>x, there are p−x+1 such windows) has at least x·y identical aligned amino acids, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if x·y is not an integer then it is rounded up to the nearest integer. The preferred pairwise alignment algorithm is the Needleman-Wunsch global alignment algorithm [18], using default parameters (e.g. with Gap opening penalty=10.0, and with Gap extension penalty=0.5, using the EBLOSUM62 scoring matrix). This algorithm is conveniently implemented in the needle tool in the EMBOSS package [19].

Within group (c), deletions or substitutions may be at the N-terminus and/or C-terminus, or may be between the two termini. Thus a truncation is an example of a deletion. Truncations may involve deletion of up to 40 (or more) amino acids at the N-terminus and/or C-terminus.

The Spb1 and GBS80 sequences in hybrid polypeptides may be derived from one or more GBS strains. For instance, SEQ ID NOs: 10 and 26 include Spb1 sequence from strain COH1 and GBS-80 sequence from strain 2603V/R.

Polypeptides

Polypeptides of the invention, or individual moieties, may, compared to SEQ ID NOs: 1, 2, 3, 4, 5, 10, 26, 34, or 36 include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may have one or more (e.g. 1, 2, 3, 4, 5, 6, 7, 8, 9, 10, etc.) single amino acid deletions relative to a reference sequence. The polypeptides may also include one or more (e.g. 1, 2, 3, 4, 5, 6, 7, 8, 9, 10, etc.) insertions (e.g. each of 1, 2, 3, 4 or 5 amino acids) relative to a reference sequence.

Polypeptides of the invention can be prepared in many ways e.g. by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. A preferred method for production of peptides <40 amino acids long involves in vitro chemical synthesis [20,21]. Solid-phase peptide synthesis is particularly preferred, such as methods based on tBoc or Fmoc [22] chemistry. Enzymatic synthesis [23] may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) [24]. Where D-amino acids are included, however, it is preferred to use chemical synthesis. Polypeptides of the invention may have covalent modifications at the C-terminus and/or N-terminus.

Polypeptides of the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides of the invention are preferably provided in purified or substantially purified form i.e. substantially free from other polypeptides (e.g. free from naturally-occurring polypeptides), particularly from other GBS or host cell polypeptides, and are generally at least about 50% pure (by weight), and usually at least about 90% pure i.e. less than about 50%, and more preferably less than about 10% (e.g. 5% or less) of a composition is made up of other expressed polypeptides.

Polypeptides of the invention may be attached to a solid support. Polypeptides of the invention may comprise a detectable label (e.g. a radioactive or fluorescent label, or a biotin label).

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains. Polypeptides of the invention can be naturally or non-naturally glycosylated (i.e. the polypeptide has a glycosylation pattern that differs from the glycosylation pattern found in the corresponding naturally occurring polypeptide).

Polypeptides of the invention may be at least 40 amino acids long (e.g. at least 40, 50, 60, 70, 80, 90, 100, 120, 140, 160, 180, 200, 220, 240, 260, 280, 300, 350, 400, 450, 500 or more). Polypeptides of the invention may be shorter than 1100 amino acids.

The invention provides a process for producing polypeptides of the invention, comprising culturing a host cell of to the invention under conditions which induce polypeptide expression. Although expression of the polypeptide may take place in a Streptococcus, the invention will usually use a heterologous host for expression. The heterologous host may be prokaryotic (e.g. a bacterium) or eukaryotic. It will usually be E. coli, but other suitable hosts include Bacillus subtilis, Vibrio cholerae, Salmonella typhi, Salmonella typhimurium, Neisseria lactamica, Neisseria cinerea, Mycobacteria (e.g. M. tuberculosis), yeasts, etc.

The invention provides a process for producing a polypeptide of the invention, wherein the polypeptide is synthesised in part or in whole using chemical means.

The invention provides a composition comprising two or more polypeptides of the invention.

Nucleic Acids

The invention also provides a nucleic acid comprising a nucleotide sequence encoding the polypeptides of the invention e.g. SEQ ID NOs: 11, 29, 31, 33, or 35. The invention also provides nucleic acid comprising nucleotide sequences having sequence identity to such nucleotide sequences. Such nucleic acids include those using alternative codons to encode the same amino acid. For example, the codon usage in the nucleic acids may be optimised to reflect codon usage in the organism in which it is intended to express the nucleic acid in order to enhance expression. Suitable approaches for codon optimisation are described, for example, in reference [25]. Nucleic acids of the invention thus include SEQ ID NOs 37 and 38 which encode the polypeptides of SEQ ID NOS: 26 and 36 but which, compared to SEQ ID NOs 29 and 35, have been optimised for expression in E. coli.

The invention also provides nucleic acid which can hybridize to these nucleic acids. Hybridization reactions can be performed under conditions of different “stringency”. Conditions that increase stringency of a hybridization reaction of widely known and published in the art. Examples of relevant conditions include (in order of increasing stringency): incubation temperatures of 25° C., 37° C., 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art [e.g. see refs 26 & 27, etc.].

The invention includes nucleic acid comprising sequences complementary to these sequences (e.g. for antisense or probing, or for use as primers).

Nucleic acid according to the invention can take various forms (e.g. single-stranded, double-stranded, vectors, primers, probes, labelled etc.). Nucleic acids of the invention may be circular or branched, but will generally be linear. Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids of the invention are preferably provided in purified or substantially purified form i.e. substantially free from other nucleic acids (e.g. free from naturally-occurring nucleic acids), particularly from other GAS or host cell nucleic acids, generally being at least about 50% pure (by weight), and usually at least about 90% pure. Nucleic acids of the invention are preferably GAS nucleic acids.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acid of the invention may be attached to a solid support (e.g. a bead, plate, filter, film, slide, microarray support, resin, etc.). Nucleic acid of the invention may be labelled e.g. with a radioactive or fluorescent label, or a biotin label. This is particularly useful where the nucleic acid is to be used in detection techniques e.g. where the nucleic acid is a primer or as a probe.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the invention includes mRNA, tRNA, rRNA, ribozymes, DNA, cDNA, recombinant nucleic acids, branched nucleic acids, plasmids, vectors, probes, primers, etc. Where nucleic acid of the invention takes the form of RNA, it may or may not have a 5′ cap.

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. Preferred vectors are plasmids. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

Where a nucleic acid is DNA, it will be appreciated that “U” in a RNA sequence will be replaced by “T” in the DNA. Similarly, where a nucleic acid is RNA, it will be appreciated that “T” in a DNA sequence will be replaced by “U” in the RNA.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T).

Nucleic acids of the invention can be used, for example: to produce polypeptides; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides.

The invention provides a process for producing nucleic acid of the invention, wherein the nucleic acid is synthesised in part or in whole using chemical means.

The invention provides vectors comprising nucleotide sequences of the invention (e.g. cloning or expression vectors) and host cells transformed with such vectors.

Immunogenic Compositions

Polypeptides of the invention are useful as active ingredients in immunogenic compositions. Such immunogenic compositions may be useful as vaccines. These vaccines may either be prophylactic (i.e. to prevent infection) or therapeutic (i.e. to treat infection), but will typically be prophylactic.

Compositions may thus be pharmaceutically acceptable. They will usually include components in addition to the antigens e.g. they typically include one or more pharmaceutical carrier(s) and/or excipient(s). A thorough discussion of such components is available in reference 28.

Compositions will generally be administered to a mammal in aqueous form. Prior to administration, however, the composition may have been in a non-aqueous form. For instance, although some vaccines are manufactured in aqueous form, then filled and distributed and administered also in aqueous form, other vaccines are lyophilised during manufacture and are reconstituted into an aqueous form at the time of use. Thus a composition of the invention may be dried, such as a lyophilised formulation.

The composition may include preservatives such as thiomersal or 2-phenoxyethanol. It is preferred, however, that the vaccine should be substantially free from (i.e. less than 5 μg/ml) mercurial material e.g. thiomersal-free. Vaccines containing no mercury are more preferred. Preservative-free vaccines are particularly preferred.

To control tonicity, it is preferred to include a physiological salt, such as a sodium salt. Sodium chloride (NaCl) is preferred, which may be present at between 1 and 20 mg/ml e.g. about 10±2 mg/ml NaCl. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a composition will generally be between 5.0 and 8.1, and more typically between 6.0 and 8.0 e.g. 6.5 and 7.5, or between 7.0 and 7.8.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Human vaccines are typically administered in a dosage volume of about 0.5 ml, although a half dose (i.e. about 0.25 ml) may be administered to children.

Immunogenic compositions of the invention may also comprise one or more immunoregulatory agents. Preferably, one or more of the immunoregulatory agents include one or more adjuvants. The adjuvants may include a TH1 adjuvant and/or a TH2 adjuvant, further discussed below.

Adjuvants which may be used in compositions of the invention include, but are not limited to:

A. Mineral-Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphosphates, orthophosphates), sulphates, etc. [e.g. see chapters 8 & 9 of ref 29], or mixtures of different mineral compounds, with the compounds taking any suitable form (e.g. gel, crystalline, amorphous, etc.), and with adsorption being preferred. The mineral containing compositions may also be formulated as a particle of metal salt [30].

Typical aluminium phosphate adjuvants are amorphous aluminium hydroxyphosphate with PO₄/Al molar ratio between 0.84 and 0.92, included at 0.6 mg Al³⁺/ml. Adsorption with a low dose of aluminium phosphate may be used e.g. between 50 and 100 μg Al³⁺ per conjugate per dose.

B. Oil Emulsions

Oil emulsion compositions suitable for use as adjuvants in the invention include squalene-water emulsions, such as MF59 [Chapter 10 of ref. 29; see also ref 31] (5% Squalene, 0.5% Tween 80, and 0.5% Span 85, formulated into submicron particles using a microfluidizer). Complete Freund's adjuvant (CFA) and incomplete Freund's adjuvant (IFA) may also be used.

C. Saponin Formulations [Chapter 22 of Ref. 29]

Saponin formulations may also be used as adjuvants in the invention. Saponins are a heterogeneous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 32. Saponin formulations may also comprise a sterol, such as cholesterol [33].

Combinations of saponins and cholesterols can be used to form unique particles called immunostimulating complexes (ISCOMs) [chapter 23 of ref. 29]. ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 33-35. Optionally, the ISCOMS may be devoid of additional detergent [36].

A review of the development of saponin based adjuvants can be found in refs. 37 & 38.

D. Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in refs. 39-44. Virosomes are discussed further in, for example, ref 45

E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref 46. Such “small particles” of 3dMPL are small enough to be sterile filtered through a 0.22 μm membrane [46]. Other non-toxic LPS derivatives include monophosphoryl lipid A mimics, such as aminoalkyl glucosaminide phosphate derivatives e.g. RC-529 [47,48].

Lipid A derivatives include derivatives of lipid A from Escherichia coli such as OM-174. OM-174 is described for example in refs. 49 & 50.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 51, 52 and 53 disclose possible analog substitutions e.g. replacement of guanosine with 2′-deoxy-7-deazaguanosine. The adjuvant effect of CpG oligonucleotides is further discussed in refs. 54-59.

The CpG sequence may be directed to TLR9, such as the motif GTCGTT or TTCGTT [60]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 61-63. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 60 & 64-66.

A particularly useful adjuvant based around immunostimulatory oligonucleotides is known as IC-31™ [67]. Thus an adjuvant used with the invention may comprise a mixture of (i) an oligonucleotide (e.g. between 15-40 nucleotides) including at least one (and preferably multiple) CpI motifs (i.e. a cytosine linked to an inosine to form a dinucleotide), and (ii) a polycationic polymer, such as an oligopeptide (e.g. between 5-20 amino acids) including at least one (and preferably multiple) Lys-Arg-Lys tripeptide sequence(s). The oligonucleotide may be a deoxynucleotide comprising 26-mer sequence 5′-(IC)₁₃-3′ (SEQ ID NO: 19). The polycationic polymer may be a peptide comprising 11-mer amino acid sequence KLKLLLLLKLK (SEQ ID NO: 20).

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref 68 and as parenteral adjuvants in ref. 69. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivatives thereof, particularly LT-K63 and LT-R72, as adjuvants can be found in refs. 70-77. A useful CT mutant is or CT-E29H [78]. Numerical reference for amino acid substitutions is preferably based on the alignments of the A and B subunits of ADP-ribosylating toxins set forth in ref. 79, specifically incorporated herein by reference in its entirety.

F. Human Immunomodulators

Human immunomodulators suitable for use as adjuvants in the invention include cytokines, such as interleukins (e.g. IL-1, IL-2, IL-4, IL-5, IL-6, IL-7, IL-12 [80], etc.) [81], interferons (e.g. interferon-γ), macrophage colony stimulating factor, and tumor necrosis factor. A preferred immunomodulator is IL-12.

G. Bioadhesives and Mucoadhesives

Bioadhesives and mucoadhesives may also be used as adjuvants in the invention. Suitable bioadhesives include esterified hyaluronic acid microspheres [82] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [83].

H. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

I. Liposomes (Chapters 13 & 14 of Ref. 29)

Examples of liposome formulations suitable for use as adjuvants are described in refs. 84-86.

J. Polyoxyethylene Ether and Polyoxyethylene Ester Formulations

Adjuvants suitable for use in the invention include polyoxyethylene ethers and polyoxyethylene esters [87]. Such formulations further include polyoxyethylene sorbitan ester surfactants in combination with an octoxynol [88] as well as polyoxyethylene alkyl ethers or ester surfactants in combination with at least one additional non-ionic surfactant such as an octoxynol [89]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

K. Polvphosphazene (PCPP)

PCPP formulations are described, for example, in refs. 90 and 91.

L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

M. Imidazoquinolone Compounds.

Examples of imidazoquinolone compounds suitable for use adjuvants in the invention include Imiquamod and its homologues (e.g. “Resiquimod 3M”), described further in refs. 92 and 93.

The invention may also comprise combinations of aspects of one or more of the adjuvants identified above. For example, the following adjuvant compositions may be used in the invention: (1) a saponin and an oil-in-water emulsion [94]; (2) a saponin (e.g. QS21)+a non-toxic LPS derivative (e.g. 3dMPL) [95]; (3) a saponin (e.g. QS21)+a non-toxic LPS derivative (e.g. 3dMPL)+a cholesterol; (4) a saponin (e.g. QS21)+3dMPL+IL-12 (optionally+a sterol) [96]; (5) combinations of 3dMPL with, for example, QS21 and/or oil-in-water emulsions [97]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and (8) one or more mineral salts (such as an aluminum salt)+a non-toxic derivative of LPS (such as 3dMPL).

Other substances that act as immunostimulating agents are disclosed in chapter 7 of ref. 29.

The use of an aluminium hydroxide and/or aluminium phosphate adjuvant is particularly preferred, and antigens are generally adsorbed to these salts. Calcium phosphate is another preferred adjuvant. Other preferred adjuvant combinations include combinations of Th1 and Th2 adjuvants such as CpG & alum or resiquimod & alum. A combination of aluminium phosphate and 3dMPL may be used.

The compositions of the invention may elicit both a cell mediated immune response as well as a humoral immune response.

Two types of T cells, CD4 and CD8 cells, are generally thought necessary to initiate and/or enhance cell mediated immunity and humoral immunity. CD8 T cells can express a CD8 co-receptor and are commonly referred to as Cytotoxic T lymphocytes (CTLs). CD8 T cells are able to recognized or interact with antigens displayed on MHC Class I molecules.

CD4 T cells can express a CD4 co-receptor and are commonly referred to as T helper cells. CD4 T cells are able to recognize antigenic peptides bound to MHC class II molecules. Upon interaction with a MHC class II molecule, the CD4 cells can secrete factors such as cytokines. These secreted cytokines can activate B cells, cytotoxic T cells, macrophages, and other cells that participate in an immune response. Helper T cells or CD4+ cells can be further divided into two functionally distinct subsets: TH1 phenotype and TH2 phenotypes which differ in their cytokine and effector function.

Activated TH1 cells enhance cellular immunity (including an increase in antigen-specific CTL production) and are therefore of particular value in responding to intracellular infections. Activated TH1 cells may secrete one or more of IL-2, IFN-γ, and TNF-β. A TH1 immune response may result in local inflammatory reactions by activating macrophages, NK (natural killer) cells, and CD8 cytotoxic T cells (CTLs). A TH1 immune response may also act to expand the immune response by stimulating growth of B and T cells with IL-12. TH1 stimulated B cells may secrete IgG2a.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

An enhanced immune response may include one or more of an enhanced TH1 immune response and a TH2 immune response.

A TH1 immune response may include one or more of an increase in CTLs, an increase in one or more of the cytokines associated with a TH1 immune response (such as IL-2, IFN-γ, and TNF-β), an increase in activated macrophages, an increase in NK activity, or an increase in the production of IgG2a. Preferably, the enhanced TH1 immune response will include an increase in IgG2a production.

A TH1 immune response may be elicited using a TH1 adjuvant. A TH1 adjuvant will generally elicit increased levels of IgG2a production relative to immunization of the antigen without adjuvant. TH1 adjuvants suitable for use in the invention may include for example saponin formulations, virosomes and virus like particles, non-toxic derivatives of enterobacterial lipopolysaccharide (LPS), immunostimulatory oligonucleotides. Immunostimulatory oligonucleotides, such as oligonucleotides containing a CpG motif, are preferred TH1 adjuvants for use in the invention.

A TH2 immune response may include one or more of an increase in one or more of the cytokines associated with a TH2 immune response (such as IL-4, IL-5, IL-6 and IL-10), or an increase in the production of IgG1, IgE, IgA and memory B cells. Preferably, the enhanced TH2 immune response will include an increase in IgG1 production.

A TH2 immune response may be elicited using a TH2 adjuvant. A TH2 adjuvant will generally elicit increased levels of IgG1 production relative to immunization of the antigen without adjuvant. TH2 adjuvants suitable for use in the invention include, for example, mineral containing compositions, oil-emulsions, and ADP-ribosylating toxins and detoxified derivatives thereof. Mineral containing compositions, such as aluminium salts are preferred TH2 adjuvants for use in the invention.

A composition may include a combination of a TH1 adjuvant and a TH2 adjuvant. Preferably, such a composition elicits an enhanced TH1 and an enhanced TH2 response, i.e., an increase in the production of both IgG1 and IgG2a production relative to immunization without an adjuvant. Still more preferably, the composition comprising a combination of a TH1 and a TH2 adjuvant elicits an increased TH1 and/or an increased TH2 immune response relative to immunization with a single adjuvant (i.e., relative to immunization with a TH1 adjuvant alone or immunization with a TH2 adjuvant alone).

The immune response may be one or both of a TH1 immune response and a TH2 response. Preferably, immune response provides for one or both of an enhanced TH1 response and an enhanced TH2 response.

The enhanced immune response may be one or both of a systemic and a mucosal immune response. Preferably, the immune response provides for one or both of an enhanced systemic and an enhanced mucosal immune response. Preferably the mucosal immune response is a TH2 immune response. Preferably, the mucosal immune response includes an increase in the production of IgA.

GBS infections can affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilised antigens.

Where a composition is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

Methods of Treatment, and Administration of the Vaccine

The invention also provides a method for raising an immune response in a mammal comprising the step of administering an effective amount of a composition of the invention. The immune response is preferably protective and preferably involves antibodies and/or cell-mediated immunity. The method may raise a booster response.

The invention also provides at least two antigens of the invention for combined use as a medicament e.g. for use in raising an immune response in a mammal.

The invention also provides the use of at least two antigens of the invention in the manufacture of a medicament for raising an immune response in a mammal.

By raising an immune response in the mammal by these uses and methods, the mammal can be protected against group B streptococcus disease and/or infection.

The invention also provides a delivery device pre-filled with an immunogenic composition of the invention.

The mammal is preferably a human. Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc.

One way of checking efficacy of therapeutic treatment involves monitoring GBS infection after administration of the compositions of the invention. One way of checking efficacy of prophylactic treatment involves monitoring immune responses, systemically (such as monitoring the level of IgG1 and IgG2a production) and/or mucosally (such as monitoring the level of IgA production), against the antigens in the compositions of the invention after administration of the composition. Typically, antigen-specific serum antibody responses are determined post-immunisation but pre-challenge whereas antigen-specific mucosal antibody responses are determined post-immunisation and post-challenge.

Another way of assessing the immunogenicity of the compositions of the present invention is to express the polypeptides recombinantly for screening patient sera or mucosal secretions by immunoblot and/or microarrays. A positive reaction between the polypeptide and the patient sample indicates that the patient has mounted an immune response to the polypeptide in question. This method may also be used to identify immunodominant antigens and/or epitopes within antigens.

The efficacy of vaccine compositions can also be determined in vivo by challenging animal models of GBS infection, e.g., guinea pigs or mice, with the vaccine compositions. Neonatal mice models are commonly used.

Compositions of the invention will generally be administered directly to a patient. Direct delivery may be accomplished by parenteral injection (e.g. subcutaneously, intraperitoneally, intravenously, intramuscularly, or to the interstitial space of a tissue), or mucosally, such as by rectal, oral (e.g. tablet, spray), vaginal, topical, transdermal or transcutaneous, intranasal, ocular, aural, pulmonary or other mucosal administration.

The invention may be used to elicit systemic and/or mucosal immunity, preferably to elicit an enhanced systemic and/or mucosal immunity.

Preferably the enhanced systemic and/or mucosal immunity is reflected in an enhanced TH1 and/or TH2 immune response. Preferably, the enhanced immune response includes an increase in the production of IgG1 and/or IgG2a and/or IgA.

Dosage can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.).

Vaccines prepared according to the invention may be used to treat both children and adults. Thus a human patient may be less than 1 year old, 1-5 years old, 5-15 years old, 15-55 years old, or at least 55 years old. Preferred patients for receiving the vaccines are the elderly (e.g. ≧50 years old, ≧60 years old, and preferably ≧65 years), the young (e.g. ≦5 years old), hospitalised patients, healthcare workers, armed service and military personnel, pregnant women, the chronically ill, or immunodeficient patients. The vaccines are not suitable solely for these groups, however, and may be used more generally in a population. Vaccines of the invention may be used for maternal immunisation.

Vaccines produced by the invention may be administered to patients at substantially the same time as (e.g. during the same medical consultation or visit to a healthcare professional or vaccination centre) other vaccines e.g. at substantially the same time as a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A-C—W135-Y vaccine), a respiratory syncytial virus vaccine, etc.

Combinations

A composition useful for immunisation may comprise a polypeptide of the invention. In addition it may include: (i) one or more further polypeptides that elicit antibody responses against GBS proteins, particularly against GBS proteins other than GBS-80 and Spb1; (ii) a capsular saccharide from GBS; and/or (iii) one or more further polypeptides that elicit antibody responses that recognise epitopes on non-GBS organisms.

A useful polypeptide for including in such compositions is a ‘GBS67’ sequence. The wild-type GBS67 sequence from serotype V strain 2603 is SEQ ID NO: 22 herein. Thus a composition may include a polypeptide comprising an amino acid sequence that (i) has at least a % identity to SEQ ID NO: 22, and/or (ii) comprises a fragment of at least b contiguous amino acids of SEQ ID NO: 22. This polypeptide sequence will, when administered to a subject, elicit an antibody response comprising antibodies that bind to wild-type GBS67 protein e.g. to the S. agalactiae protein having amino acid sequence SEQ ID NO: 22.

Wild-type GBS67 contains a C-terminus transmembrane region which may be removed e.g. to give SEQ ID NO: 23. It also contains amino acid motifs indicative of a cell wall anchor (LPXTG and IPMTG). In some recombinant host cell systems, it may be preferable to remove this motif to facilitate secretion from the host cell. Accordingly, in one preferred fragment of GBS67 for use in the invention, the transmembrane and the cell wall anchor motif are removed (SEQ ID NO: 24). Alternatively, in some recombinant host cell systems, it may be preferable to use the cell wall anchor motif to anchor the recombinantly expressed polypeptide to the cell wall. The extracellular domain of the expressed polypeptide may be cleaved during purification or the recombinant polypeptide may be left attached to either inactivated host cells or cell membranes in the final composition.

Three pilin motifs, containing conserved lysine residues have been identified in GBS67. Conserved lysine residues are at amino acid residues 478 and 488, at amino acid residues 340 and 342, and at amino acid residues 703 and 717. The pilin sequences, in particular the conserved lysine residues, are thought to be important for the formation of oligomeric, pilus-like structures of GBS67. Preferred fragments of GBS 67 include at least one conserved lysine residue. Two E boxes containing conserved glutamic residues have also been identified in GBS67. Preferred fragments of GBS 67 include at least one conserved glutamic acid residue. GBS67 contains several regions predicted to form alpha helical structures. Such alpha helical regions are likely to form coiled-coil structures and may be involved in oligomerization of GBS67. GBS67 also contains a region which is homologous to the Cna_B domain of the S. aureus collagen-binding surface protein (pfam05738). This may form a beta sandwich structure. GBS67 contains a region which is homologous to a von Willebrand factor (vWF) type A domain

Capsular saccharides may be included from any of GBS serotypes Ia, Ib, II, III, IV, V, VI, VII, & VIII. Including a saccharide from one or more of serotypes Ia, Ib, II, III & V is useful. The capsular saccharides of each of these five serotypes include: (a) a terminal N-acetyl-neuraminic acid (NeuNAc) residue (commonly referred to as sialic acid), which in all cases is linked 2→3 to a galactose residue; and (b) a N-acetyl-glucosamine residue (GlcNAc) within the trisaccharide core.

Saccharides used according to the invention may be in their native form, or may have been modified. For example, the saccharide may be shorter than the native capsular saccharide, or may be chemically modified. For instance, the saccharide may be de-O-acetylated (partially or fully) or de-N-acetylated (partially or fully). Another possible modification is the removal of sialic acid residues from the saccharide, such as side-chain terminal sialic acids [98].

Saccharides will typically be conjugated to a carrier protein. In general, covalent conjugation of saccharides to carriers enhances the immunogenicity of saccharides as it converts them from T-independent antigens to T-dependent antigens, thus allowing priming for immunological memory.

Preferred carrier proteins are bacterial toxins, such as diphtheria or tetanus toxins, or toxoids or mutants thereof. These are commonly used in conjugate vaccines. The CRM₁₉₇ diphtheria toxin mutant is particularly preferred [99]. Other suitable carrier proteins include the N. meningitidis outer membrane protein complex [100], synthetic peptides [101,102], heat shock proteins [103,104], pertussis proteins [105,106], cytokines [107], lymphokines [107], hormones [107], growth factors [107], artificial proteins comprising multiple human CD4⁺ T cell epitopes from various pathogen-derived antigens [108] such as N19 [109], protein D from H. influenzae [110-112], pneumolysin [113] or its non-toxic derivatives [114], pneumococcal surface protein PspA [115], iron-uptake proteins [116], toxin A or B from C. difficile [117], recombinant Pseudomonas aeruginosa exoprotein A (rEPA) [118], etc.

Details of conjugates suitable for use with the invention are given in reference 119.

General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Antibodies will generally be specific for their target. Thus they will have a higher affinity for the target than for an irrelevant control protein, such as bovine serum albumin.

Identity between polypeptide sequences is preferably determined by the Smith-Waterman homology search algorithm as implemented in the MPSRCH program (Oxford Molecular), using an affine gap search with parameters gap open penalty=12 and gap extension penalty=1.

### BRIEF DESCRIPTION OF THE DRAWINGS

FIG. 1 shows the amplification and joining strategy for creating the GBS80-Spb1 hybrid, and

FIGS. 2 and 3 show analysis of this polypeptide after expression.

FIG. 4 illustrates the Spb1-GBS80 hybrid. FIG. 5 shows its expression as two bands, and FIG. 6 shows the loss of the lower-MW band after the second RBS was removed.

FIGS. 7-10 show analysis of the hybrid after purification.

FIG. 11 shows analysis of the hybrid after storage under different conditions. In each gel the lanes are, from left to right: markers; protein stored at −20° C., 4° C., 25° C., 37° C. in the absence of protease inhibitors; markers; protein stored at −20° C., 4° C., 25° C., 37° C. in the presence of protease inhibitors.

### MODES FOR CARRYING OUT THE INVENTION

GBS-80 and Spb1 were identified as two GBS proteins of interest for immunisation purposes, and it was decided to express them as a hybrid polypeptide.

Hybrid polypeptide Comprising GBS-80 and Spb1: Expression

Sequences coding for GBS-80 and Spb1 were amplified from the genomes of 2603 and COH1 strains, respectively. Oligonucleotide primers o1 and o2 were used to amplify GBS-80 (PCR A, see FIG. 1), while primers o3 and o4 were used to amplify Spb1 (PCR B):

| Primer | o1 | o2 | o3 | o4 |
|---|---|---|---|---|
| SEQ ID | 13 | 14 | 15 | 16 |

A NheI restriction site was introduced in o1, and a XhoI restriction site was introduced in o4. Primers o2 and o3 contained the DNA sequence coding for the linker (Gly₄Ser)₃ (SEQ ID NO: 6).

PCR A and B were used to obtain PCR product C (the ORF coding for the fusion GBS-80-linker-Spb1) using PCR A and PCR B as templates. PCR C was digested with NheI-XhoI and introduced into the plasmid pET-21b previously digested with the same restriction enzymes.

The resulting nucleic acid sequence was Nhe-GBS80-(Gly₄-Ser)₃-Sac-SPB1-Xho.

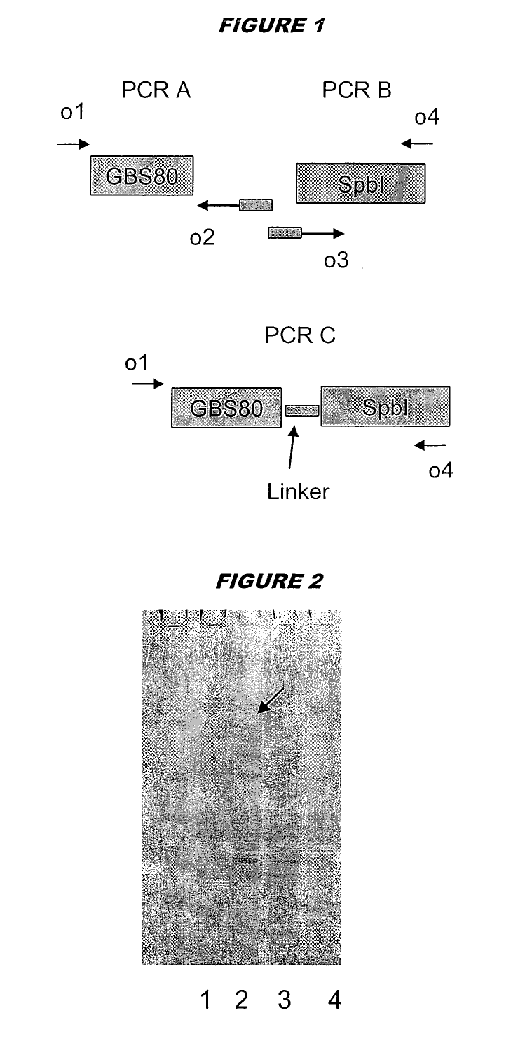

E. coli BL21 (DE3) clones containing the plasmid were checked for the expression of the recombinant polypeptide. The expression of the polypeptide was induced by addition of IPTG (0.2 mM) to the culture during exponential growth phase. FIG. 2 shows the corresponding SDS-PAGE of non induced bacteria total extract (1), induced E. coli total extracts (2), induced soluble fraction (3) and induced insoluble fraction (4). The arrow indicates the expected molecular weight fusion polypeptide. A very low amount of polypeptide can be observed.

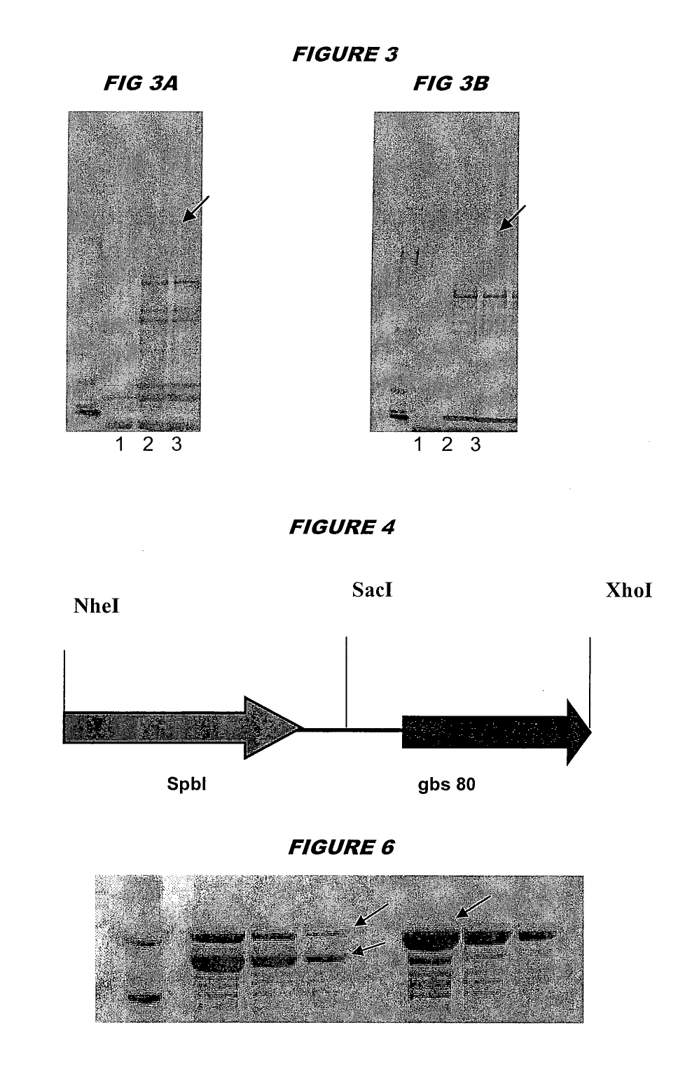

In order to confirm the data obtained, the same bacterial extracts were loaded in a second SDS PAGE and analysed by Western Blot using mouse sera anti-Spb1 (FIG. 3A) and anti-GBS80 (3B). Non induced total extracts (1), IPTG induced total (2) and soluble (3) extracts were loaded. No bands of the expected molecular weight of the fusion could be observed. Instead, a ladder of bands reacting either with anti-GBS80 or with anti-Spb1 could be detected, indicating that this fusion polypeptide is degraded in E. coli.

Thus this approach to co-expression of GBS-80 and Spb1 was abandoned.

Reverse Hybrid polypeptide: Expression

In an attempt to express GBS-80 and Spb1 as a hybrid polypeptide but without the observed degradation, their positions in the hybrid were reversed. Thus Spb1 was placed to the N-terminus of GBS-80, constructed as shown in FIG. 4 (NheI-SPB1-(Gly₄-Ser)₃-SacI-GBS80-XhoI). The coding sequence was SEQ ID NO: 11, encoding SEQ ID NO: 10 (consisting of Met-Ala-Ser i.e. SEQ ID NO: 8; then residues 30-467 of SEQ ID NO: 1 i.e. SEQ ID NO: 2; then linker SEQ ID NO: 7; then residues 38-520 of SEQ ID NO: 3 i.e. SEQ ID NO: 4). This polypeptide is referred to hereafter as RH1.

Unlike the previous attempt to express the two polypeptides in hybrid form in E. coli, this reversed order gave good levels of expressed polypeptide in the cells' soluble fraction. As shown in FIG. 5, however, two polypeptide bands were visible—one at 90 kDa and one at 102 kDa.

Investigation of the two bands my mass spectrometry revealed that the 90 kDa band had the same sequence as the 102 kDa band, but starting at residue Met-162. This finding suggested that a nucleotide sequence shortly upstream of Met-162 was acting as a second ribosome binding site, thus giving an alternative translation start position. The sequence upstream of the start codon GGAGCTGTGATTATG (SEQ ID NO: 17) was mutated to GGTGCTGTGATTATG (SEQ ID NO: 18) without altering the encoded amino acid. The nucleotide sequence of the reverse hybrid protein RH1 containing the modified Sbp1 (i.e. Spb_{ΔRBS.}-GBS80) is given in SEQ ID NO: 31. When expressed in E. coli the 102 kDa polypeptide band became much more prominent while the 90 kDa band decreased disappeared FIG. 6). Moreover, the recombinant polypeptide yield increased from 2.25 mg/L to 3.48 mg/L.

Thus removal of the second RBS substantially improved expression of the hybrid polypeptide. The modified Spb1 is referred to hereafter as Spb1_{ΔRBS.}

Further work on expression confirmed that the recombinant polypeptide Spb1_{ΔRBS.}-GBS80 could be efficiently expressed in E. coli BL21(DE3) from vector pET24b using a simple batch process in BSE-free complex medium. The quantity of inductor needed for high expression was found to be low (0.25 mM IPTG), and the polypeptide was found to accumulate at an acceptable level of saturation. No consistent degradation was observed either at early induction or after 9 hours of induction. Growth rate of the strain was affected only minimally by induction. Saturation of specific product was obtained after 5 hours of induction and was then maintained until harvest. Volumetric productivity increased until harvest, and concentrations of the polypeptide reached 0.49 g/L.

Growth was tested in the absence of antibiotic (kanamycin). Expression levels were not affected and plasmid content remained good until induction (43 plasmids/genome at the moment of induction). After induction an increase in PCN was seen. Hence, no kanamycin is needed for stabilising the system during this process.

In further expression experiments, the amino acid linker in this polypeptide was replaced by SEQ ID NO: 25, to give a polypeptide with amino acid sequence SEQ ID NO: 26. Primers for amplifying the Spb1 fragment and adding the linker were SEQ ID NOs: 27 and 28. A gene (SEQ ID NO: 29) encoding this polypeptide was expressed in E. coli in a pET-24b vector (Novagen™). This polypeptide is referred to hereafter as RH2.

During subsequent experiments, the components of RH1 and RH2 were re-sequenced and it was realised that a spontaneous nucleotide substitution from CAA to AAA had occurred at the codon corresponding to codon 12 of the Sbp1 nucleotide sequence of SEQ ID NO:2, resulting in an amino acid substitution from Q to K in Sbp1 at this position. The amino acid sequence for the mutant Sbp1 fragment resulting from this substitution is shown in SEQ ID NO:32.

The nucleotide sequence of the modified RH1 containing Spb1_{ΔRBS} and the point mutation is shown in SEQ ID NO:33. The encoded amino acid sequence of the modified RH1 polypeptide is shown in

SEQ ID NO:34. The nucleotide sequence of the modified RH2 containing Spb1_{ΔRBS} and the point mutation is shown in SEQ ID NO:35. The encoded amino acid sequence of the modified RH2 polypeptide is shown in SEQ ID NO:36. The modified reverse hybrid polypeptides shown in SEQ ID NO:34 and SEQ ID NO:36 are termed RH1′ and RH2′ respectively.

Since it is not known when the nucleotide substitution in the Spb1 component occurred, some of the experiments described in this example as being conducted with RH1 may actually have been conducted with RH1′. The presence of this substitution does not appear to alter the properties of the reverse hybrid polypeptides. Further work on expression has confirmed that both RH2 and RH2′ can be expressed in E. coli in a robust, reproducible process.

The nucleotide sequences encoding the RH2 and RH2′ hybrid polypeptides were optimised with the aim of improving expression in E. coli. Codons in the nucleotide sequences that are rare in E. coli were replaced with codons encoding the same amino acid that are common in E. coli using GeneOptimzer® technology. The optimised RH2 nucleotide sequence is given in SEQ ID NO:37. The optimised RH2′ nucleotide sequence is given in SEQ ID NO: 38.

Reverse Hybrid polypeptide: Purification

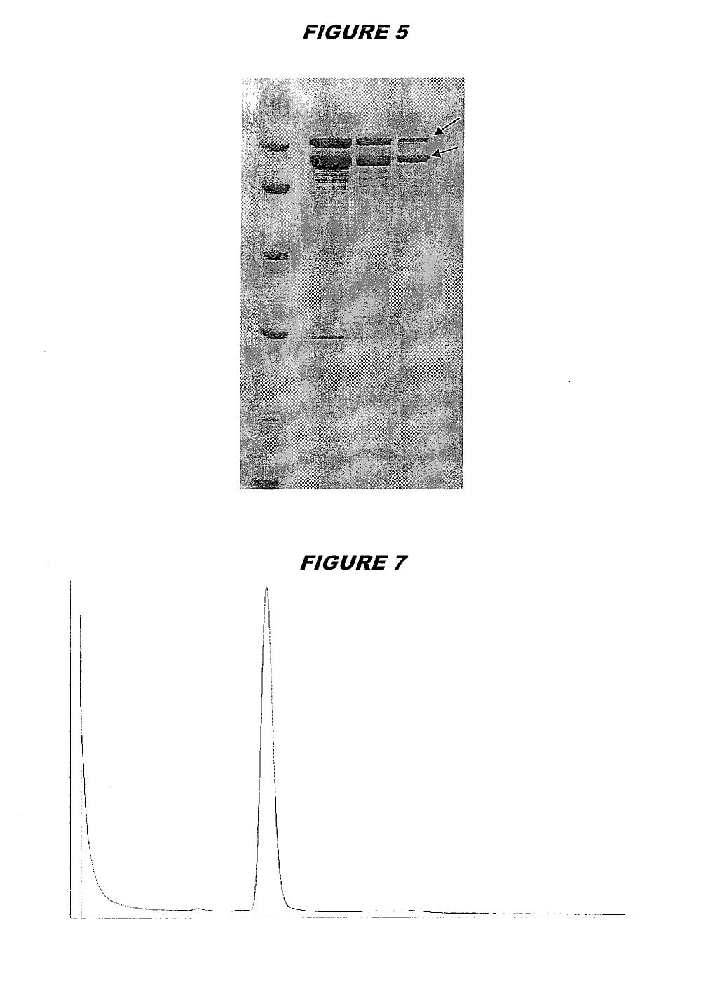

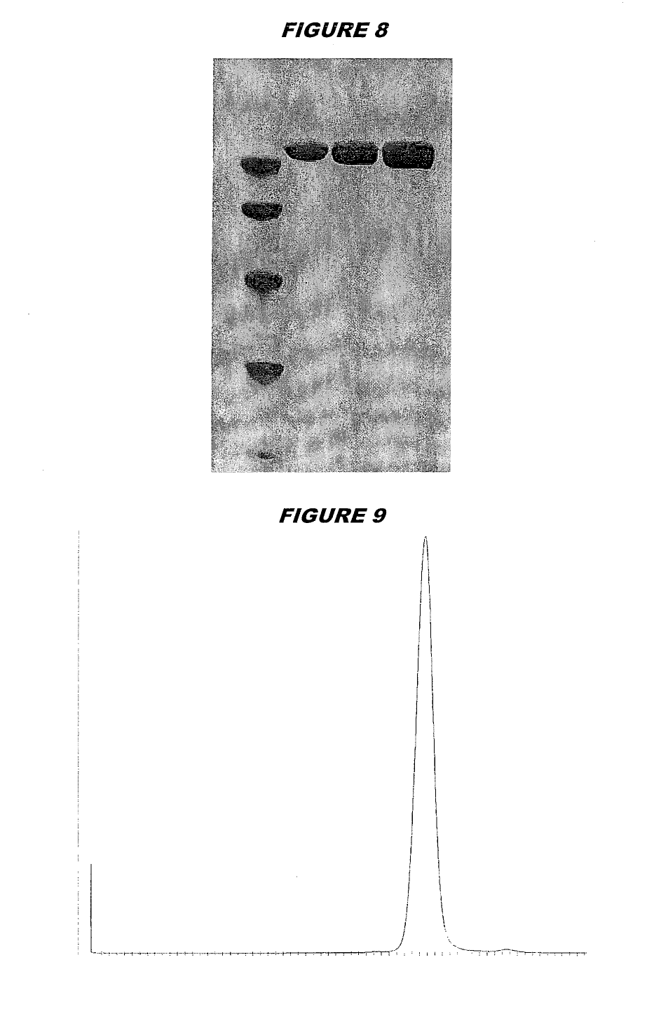

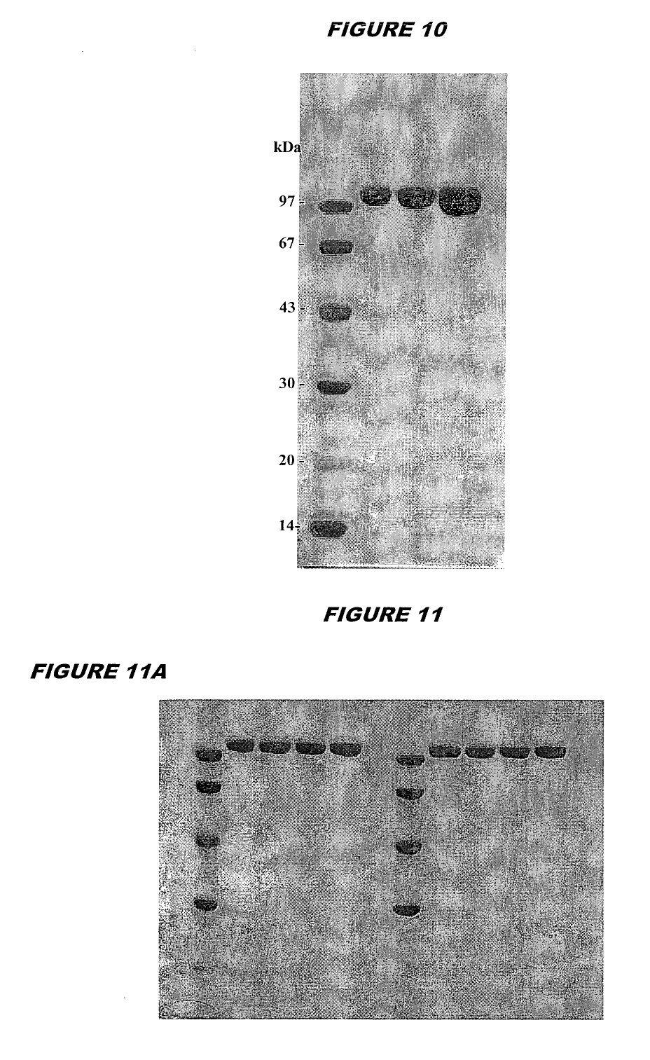

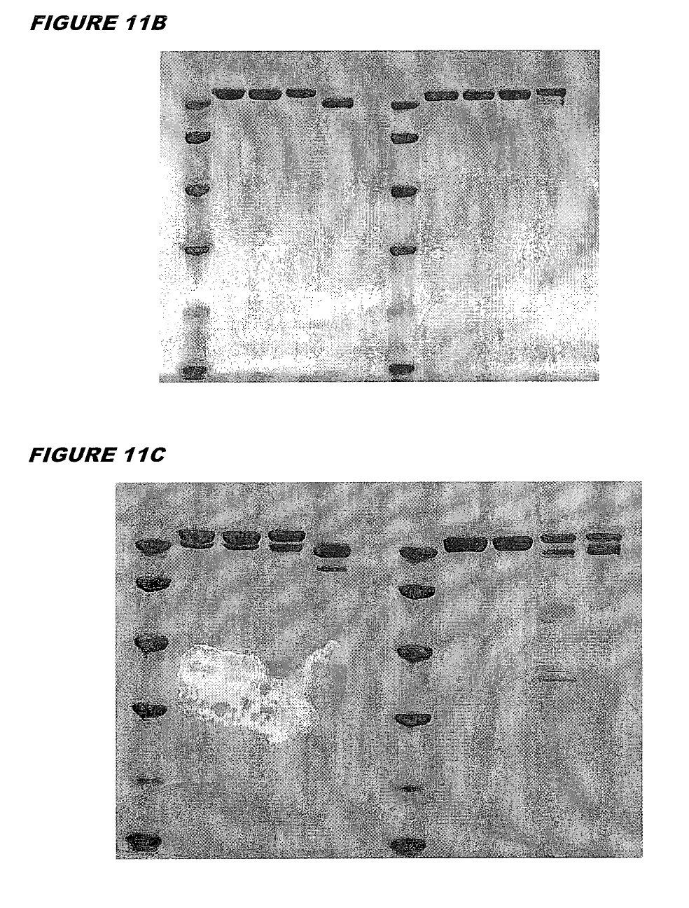

Various techniques were used to purify the reverse hybrid protein RH1, including Q-Sepharose FF, Hydroxyapatite Macro-Prep Ceramic, Phenyl Sepharose FF, HiTrap Chelating HP, and Superdex 200. Yields ranged from 25% to 37% in initial studies. FIG. 7 shows the output of gel filtration using Superdex 200. FIG. 8 shows SDS-PAGE of the purified protein at 2.5 μg, 5 μg or 10 μg. FIGS. 9 and 10 show similar data for a later lot where 47 grams of wet biomass led to a yield of 0.97 mg of recombinant protein per gram of wet biomass.

Similar techniques were used to purify the reverse hybrid proteins RH2 and RH2′. Volumetric productivity of RH2 after purification was 10 mg/L.

Reverse Hybrid polypeptide: Immunogenicity

RH1 was used to immunise animals in a lethal challenge model. The two separate antigens were usually also tested either alone or in admixture. Various different challenge strains were used: COH1; A909; M781; CJB111; JMU071; M732; 6213, JM9130013. PBS was used as a negative control.

Results of different experiments were as follows, showing % survival rates:

| Challenge | Spb1 | GBS-80 | Spb1 + | | |
|---|---|---|---|---|---|
| strain | alone | alone | GBS-80 mix | Hybrid | Control |
| COH1 | 82 | 47 | 100 | 100 | 0 |
| A909 | 90 | — | 98 | 77 | 27 |
| M781 | 96 | — | 100 | 90 | 5 |
| CJB111 | 20 | 70 | 50 | 61 | 3 |
| JMU071 | 0 | — | 0 | 53 | 13 |

| Challenge strain | Spb1 alone | GBS-80 alone | Hybrid | Control |
|---|---|---|---|---|
| COH1 | 40 | 77 | 77 | 10 |
| CJB111 | — | 67 | 65 | 17 |
| M732 | 61 | 41 | 89 | 20 |
| M781 | 77 | — | 76 | 20 |
| 6213 | 80 | 5 | 87 | 3 |

Freund's adjuvant used

| Challenge strain | Spb1 alone | GBS-80 alone | Hybrid | Control |
|---|---|---|---|---|
| COH1 | 73 | 92 | 75 | 2 |
| A909 | 87 | — | 68 | 24 |
| CJB111 | — | 44 | 42 | 21 |
| M732 | 93 | 77 | 97 | 43 |
| 6213 | 72 | — | 85 | 0 |

Alum adjuvant used

| Challenge strain | Spb1 + GBS-80 mix | Hybrid | Control |
|---|---|---|---|
| A909 | 100 | 92 | 5 |
| COH1 | 92 | 100 | 17 |
| CJB111 | 78 | 80 | 10 |
| M732 | 87 | 93 | 28 |

Freund's adjuvant used

| Challenge strain | Spb1 + GBS-80 mix | Hybrid | Control |
|---|---|---|---|
| COH1 | 88 | 83 | 23 |
| A909 | 90 | 100 | 27 |
| CJB111 | 55 | 68 | 27 |
| M732 | 100 | 96 | 37 |
| 6213 | 88 | 78 | 27 |
| M781 | 90 | 98 | 22 |
| JM19130013 | — | 85 | 35 |

MF59 + CpG adjuvant used

RH2 was also used to immunise animals in a lethal challenge model and the results compared to immunization with RH1. The Spb1 and GBS80 antigens were also tested in admixture. Strains COH1 A909 and CJB 111 were used for the challenge. PBS was used as a negative control.

| | | RH2 | RH1 | |
|---|---|---|---|---|
| Challenge strain | Spb1 + GBS-80 mix | Hybrid | Hybrid | Control |
| CJB111 | 44 | 74 | 56 | 24 |
| COH1 | 86 | 83 | 88 | 36 |
| A909 | 87 | 76 | 82 | 52 |

Freund's adjuvant used

Thus in situations where a GBS80 and Spb1 fail to offer high levels of protection, either alone or in simple combination, the hybrid proteins perform well.

It will be understood that the invention has been described by way of example only and modifications may be made whilst remaining within the scope and spirit of the invention.

### REFERENCES (THE CONTENTS OF WHICH ARE HEREBY INCORPORATED BY REFERENCE)

[1] Maione et al. (2005) Science 309:148-50.

[2] Tettelin et al. (2006) Expert Rev Vaccines 5:687-94.

[3] Geysen et al. (1984) PNAS USA 81:3998-4002.

[4] Carter (1994) Methods Mol Biol 36:207-223.

[5] Jameson, B A et al. 1988, CABIOS 4(1):181-186.

[6] Raddrizzani & Hammer (2000) Brief Bioinform 1(2):179-189.

[7] De Lalla et al. (1999) J. Immunol. 163:1725-1729.

[8] Brusic et al. (1998) Bioinformatics 14(2):121-130

[9] Meister et al. (1995) Vaccine 13(6):581-591.

[10] Roberts et al. (1996) AIDS Res Hum Retroviruses 12(7):593-610.

[11] Maksyutov & Zagrebelnaya (1993) Comput Appl Biosci 9(3):291-297.

[12] Feller & de la Cruz (1991) Nature 349(6311):720-721.

[13] Hopp (1993) Peptide Research 6:183-190.

[14] Welling et al. (1985) FEBS Lett. 188:215-218.

[15] Davenport et al. (1995) Immunogenetics 42:392-397.

[16] WO02/34771.

[17] WO2006/082527.

[18] Needleman & Wunsch (1970) J. Mol. Biol. 48, 443-453.

[19] Rice et al. (2000) Trends Genet 16:276-277.

[20] Bodanszky (1993) Principles of Peptide Synthesis (ISBN: 0387564314).

[21] Fields et al. (1997) Meth Enzymol 289: Solid-Phase Peptide Synthesis. ISBN: 0121821900.

[22] Chan & White (2000) Fmoc Solid Phase Peptide Synthesis. ISBN: 0199637245.

[23] Kullmann (1987) Enzymatic Peptide Synthesis. ISBN: 0849368413.

[24] Ibba (1996) Biotechnol Genet Eng Rev 13:197-216.

[25] Puigbo et al, Nucleic Acids Research, 2007, 1-6

[26] U.S. Pat. No. 5,707,829

[27] Current Protocols in Molecular Biology (F. M. Ausubel et al. eds., 1987) Supplement 30.

[28] Gennaro (2000) Remington: The Science and Practice of Pharmacy. 20th edition, ISBN: 0683306472.

[29] Vaccine Design . . . (1995) eds. Powell & Newman. ISBN: 030644867X. Plenum.

[30] WO00/23105.

[31] WO90/14837.

[32] U.S. Pat. No. 5,057,540.

[33] WO96/33739.

[34] EP-A-0109942.

[35] WO96/11711.

[36] WO00/07621.

[37] Barr et al. (1998) Advanced Drug Delivery Reviews 32:247-271.

[38] Sjolanderet et al. (1998) Advanced Drug Delivery Reviews 32:321-338.

[39] Niikura et al. (2002) Virology 293:273-280.

[40] Lenz et al. (2001) J Immunol 166:5346-5355.

[41] Pinto et al. (2003) J Infect Dis 188:327-338.

[42] Gerber et al. (2001) J Virol 75:4752-4760.

[43] WO03/024480.

[44] WO03/024481.

[45] Gluck et al. (2002) Vaccine 20:B10-B16.

[46] EP-A-0689454.

[47] Johnson et al. (1999) Bioorg Med Chem Lett 9:2273-2278.

[48] Evans et al. (2003) Expert Rev Vaccines 2:219-229.

[49] Meraldi et al. (2003) Vaccine 21:2485-2491.

[50] Pajak et al. (2003) Vaccine 21:836-842.

[51] Kandimalla et al. (2003) Nucleic Acids Research 31:2393-2400.

[52] WO02/26757.

[53] WO99/62923.

[54] Krieg (2003) Nature Medicine 9:831-835.

[55] McCluskie et al. (2002) FEMS Immunology and Medical Microbiology 32:179-185.

[56] WO98/40100.

[57] U.S. Pat. No. 6,207,646.

[58] U.S. Pat. No. 6,239,116.

[59] U.S. Pat. No. 6,429,199.

[60] Kandimalla et al. (2003) Biochemical Society Transactions 31 (part 3):654-658.

[61] Blackwell et al. (2003) J Immunol 170:4061-4068.

[62] Krieg (2002) Trends Immunol 23:64-65.

[63] WO01/95935.

[64] Kandimalla et al. (2003) BBRC 306:948-953.

[65] Bhagat et al. (2003) BBRC 300:853-861.

[66] WO03/035836.

[67] Schellack et al. (2006) Vaccine 24:5461-72.

[68] WO95/17211.

[69] WO98/42375.

[70] Beignon et al. (2002) Infect Immun 70:3012-3019.

[71] Pizza et al. (2001) Vaccine 19:2534-2541.

[72] Pizza et al. (2000) Int J Med Microbiol 290:455-461.

[73] Scharton-Kersten et al. (2000) Infect Immun 68:5306-5313.

[74] Ryan et al. (1999) Infect Immun 67:6270-6280.

[75] Partidos et al. (1999) Immunol Lett 67:209-216.

[76] Peppoloni et al. (2003) Expert Rev Vaccines 2:285-293.

[77] Pine et al. (2002) J Control Release 85:263-270.

[78] Tebbey et al. (2000) Vaccine 18:2723-34.

[79] Domenighini et al. (1995) Mol Microbiol 15:1165-1167.

[80] WO99/40936.

[81] WO99/44636.

[82] Singh et al] (2001) J Cont Release 70:267-276.

[83] WO99/27960.

[84] U.S. Pat. No. 6,090,406.

[85] U.S. Pat. No. 5,916,588.

[86] EP-A-0626169.

[87] WO99/52549.

[88] WO01/21207.

[89] WO01/21152.

[90] Andrianov et al. (1998) Biomaterials 19:109-115.

[91] Payne et al. (1998) Adv Drug Delivery Review 31:185-196.

[92] Stanley (2002) Clin Exp Dermatol 27:571-577.

[93] Jones (2003) Curr Opin Investig Drugs 4:214-218.

[94] WO99/11241.

[95] WO94/00153.

[96] WO98/57659.

[97] European patent applications 0835318, 0735898 and 0761231.

[98] WO2006/050341.

[99] Research Disclosure, 453077 (January 2002)

EP-A-0372501.

EP-A-0378881.

EP-A-0427347.

WO93/17712

WO94/03208.

WO98/58668.

EP-A-0471177.

WO91/01146

Falugi et al. (2001) Eur J Immunol 31:3816-3824.

Baraldo et al. (2004) Infect Immun 72(8):4884-7.

EP-A-0594610.

Ruan et al. (1990) J Immunol 145:3379-3384.

WO00/56360.

Kuo et al. (1995) Infect Immun 63:2706-13.

Michon et al. (1998) Vaccine. 16:1732-41.

WO02/091998.

WO01/72337

WO00/61761.

WO00/33882

WO2006/082530.

1. A polypeptide comprising amino acid sequence NH₂—W—X-L-Y—Z—CO₂H, wherein: X is a Spb1 sequence; L is an optional linker; Y is a GBS-80 sequence; W is an optional N-terminal sequence; and Z is an optional C-terminal sequence.

2. The polypeptide of claim 1, wherein the Spb1 sequence will, when administered to a subject, elicit an antibody response comprising antibodies that bind to SEQ ID NO:1.

3. The polypeptide of claim 1 wherein the Spb1 sequence comprises an amino acid sequence (i) having at least 80% identity to SEQ ID NO:2, and/or (ii) comprising a fragment of at least 7 amino acids of SEQ ID NO:2.

4. The polypeptide of claim 1 wherein the GBS-80 sequence will, when administered to a subject, elicit an antibody response comprising antibodies that bind to SEQ ID NO:3.

5. The polypeptide of claim 1 wherein the GBS-80 sequence comprises an amino acid sequence (i) having at least 80% identity to SEQ ID NO:4, and/or (ii) comprising a fragment of at least 7 amino acids of SEQ ID NO:4.

6. The polypeptide of claim 1 which comprises L and wherein L is 2-40 amino acids long.

7. The polypeptide of claim 1 which comprises W and wherein W is 2-40 amino acids long.

8. The polypeptide of claim 1 which comprises Z and wherein Z is 2-40 amino acids long.

9. The polypeptide of claim 1 comprising SEQ ID NO:10, SEQ ID NO:26, SEQ ID NO:34 or SEQ ID NO:36.

10. A polypeptide comprising an amino acid sequence having at least 70% sequence identity to SEQ ID NO:10 0, to SEQ ID NO:26, to SEQ ID NO:34 or to SEQ ID NO:36.

11. Nucleic acid encoding the polypeptide of claim 1.

12. The nucleic acid of claim 11, comprising SEQ ID NO:11, SEQ ID NO:29, SEQ ID NO:31, SEQ ID NO:33, SEQ ID NO:35, SEQ ID NO:37 or SEQ ID NO:38.

13. A method for raising an immune response in a mammal comprising the step of administering an effective amount of a composition comprising the polypeptide of claim 1.

14. Nucleic acid encoding a Spb1 sequence, wherein the Spb1 sequence includes a N-terminus methionine start codon and an internal methionine codon, and wherein the nucleic acid does not include a GGAG 4-mer nucleotide sequence in the 15 nucleotides upstream of the internal methionine codon.

15. A pharmaceutical composition comprising a polypeptide according to claim 1 and at least one of: (i) one or more further polypeptides that elicit antibody responses against a GBS protein other than GBS80 and Spb1; and/or (ii) a capsular saccharide from GBS.

16. The composition of claim 15, comprising a polypeptide that, when administered to a subject, elicits an antibody response comprising antibodies that bind to SEQ ID NO: 22.

17. The composition of claim 15 comprising a conjugated saccharide from one or more of GBS serotypes Ia, Ib, II, III & V.
